SEQUENCE LISTING (1 sequence)

SEQ ID NO: 1 - protein, 583 aa, Aspergillus niger
SNGIEASLLTDPKDVSGRTVDYIIAGGGLTGLTTAARLTENPNISVLVIESGSYESDRGPIIEDLNAYGDIFGSSVDHAYETVELATNNQTALIRSGNGLGGSTLVNGGTWTRPHKAQVDSWETVFGNEGWNWDNVAAYSLQAERARAPNAKQIAAGHYFNASCHGVNGTVHAGPRDTGDDYSPIVKALMSAVEDRGVPTKKDFGCGDPHGVSMFPNTLHEDQVRSDAAREWLLPNYQRPNLQVLTGQYVGKVLLSQNGTTPRAVGVEFGTHKGNTHNVYAKHEVLLAAGSAVSPTILEYSGIGMKSILEPLGIDTVVDLPVGLNLQDQTTATVRSRITSAGAGQGQAAWFATFNETFGDYSEKAHELLNTKLEQWAEEAVARGGFHNTTALLIQYENYRDWIVNHNVAYSELFLDTAGVASFDVWDLLPFTRGYVHILDKDPYLHHFAYDPQYFLNELDLLGQAAATQLARNISNSGAMQTYFAGETIPGDNLAYDADLSAWTEYIPYHFRPNYHGVGTCSMMPKEMGGVVDNAARVYGVQGLRVIDGSIPPTQMSSHVMTVFYAMALKISDAILEDYASMQ

(19) United States
(12) Patent Application Publication
(10) Pub. No.: US 2009/0127112 A1
(43) Pub. Date: May 21, 2009

(54) Biologically active metal-coated proteins

(75) Inventors: Amihay Freeman, Ben-Shemen (IL); Yosi Shacham-Diamand, Zikhron-Yaakov (IL); Sefi Vernick, Tel-Aviv (IL); Hila Moscovich-Dagan, Hod-HaSharon (IL)

Correspondence Address: MARTIN D. MOYNIHAN d/b/a PRTSI, INC., P.O. BOX 16446, ARLINGTON, VA, 22215, US

(73) Assignee: Ramot At Tel Aviv University Ltd., Tel-Aviv (IL)

(21) Appl. No.: 11/920,689

(22) PCT Filed: May 18, 2006
(86) PCT No.: PCT/IL2006/000587

§ 371 Date: Nov. 19, 2007

Related U.S. Application Data
(60) Provisional application No. 60/682,006, filed on May 18, 2005.

(51) Int. Cl. G01N 33/487; C12N 9/04; A61K 9/00; A61K 38/44

(52) U.S. Cl. 204/403.01; 435/190; 424/400; 514/6; 428/615; 205/777.5

(57) ABSTRACT

Metal-coated proteins, being dissolvable or suspendable in aqueous media and/or retaining a biological activity of the protein, a process and intermediates for preparing same are disclosed. Further disclosed are a pharmaceutical composition containing and a method of treating bacterial and fungal infections utilizing biologically active metal-coated proteins. Conductive elements, electronic circuits containing same, electrodes and biosensor systems utilizing same, and imaging probes, all containing the metal-coated proteins, are also disclosed.

### FIELD AND BACKGROUND OF THE INVENTION

The present invention relates to novel biologically active composites and, more particularly, to biologically active metal-coated proteins and cells. The present invention further relates to processes and intermediates for the preparation of such composites, and to uses thereof in, for example, pharmaceuticals, biosensors, imaging, nuclear medicine and electronic devices.

The great potential in the overlap between nanotechnology and biotechnology brought to the development of hybrid systems and components which combine the molecular size scale, solubility, selectivity of pattern recognition and biochemical activity of biological molecules, particles and microorganisms, jointly referred to as biological entities, such as peptides, oligonucleotides, proteins, viruses and cells, with electric, magnetic and photoelectric characteristics of nanoparticles such as conductive and/or magnetic metal nanoparticles and nanocrystals which exhibit unique spectral and semi-conductive characteristics. Systems and components having these attributes are designed for integration in many applications such as biosensors, biomarkers, targeted pharmaceuticals and diagnostic tools, nanowires, nanoelectronics and nanodevices.

Combining the biological activity of biological entities with electrical conductivity is one of the most promising avenues in nanotechnology. Nanocircuitry depends on the availability of highly efficient and precisely manufactured nanowires, and hence poses strict requirements of the size and regularity of such nanowires. The utilization of protein and/or DNA templates, self-assembled protein fibers, nanotubular peptide-based structures and layers, and variable length and self-hybridizable DNA chains may offer a viable solution to these requirements. Examples of hybrid systems of metal oxides or conductive metals and proteins which are known in the art include multilayered arrays of conjugates of cytochrome C and TiO₂ nanoparticles, and mica surface coated with streptavidin-labeled gold nanoparticles conjugated to biotin-labeled viral DNA. Such hybrids can be used as labeling elements in imaging and optical analysis techniques and systems.

The attachment of magnetic nanoparticles to biological entities have been used, for example, to form affinity chromatography systems based on magnetic antigen/antibody affinity pairs, for gene identification using magnetically labeled DNA hybrid systems, and for electrochemical switches. Ferritin, which is considered as the iron storage protein in the body, having 20% of its mass as iron, was used to form magnetic cobalt/platinum nanoparticles in its inner cavity.

One particular analytical branch which can beneficially utilize metal-protein hybrids is the field of biosensors, and in particular enzyme-coated electrodes for ultra-sensitive amperometric detection of various analyte at low overpotentials. Biosensors such as those disclosed, for example, in U.S. Pat. Nos. 5,723,345 6,218,134, 6,773,564, 6,776,888, 6,982,027, 6,984,307, 6,942,770 and Japanese Patent No. 2517153, are analytical devices which convert a biological response into an electrical signal, and thus can quantitatively and qualitatively determine a specific biochemical analyte in a sample. Biosensors can be produced by forming an electrode system having a working electrode (also referred to in the art as “measuring electrode”) and a reactive layer applied thereon, which includes, for example, a redox enzyme that reacts with the biochemical analyte. When the reactive layer contacts a sample that contains the analyte, the analyte is catalytically oxidized by the redox enzyme. The catalytic reaction is typically performed in the presence of an electron-transfer mediator, which is reduced upon the oxidation reaction and is then re-oxidized electrochemically. The concentration of the analyte in the sample is determined upon the recorded oxidation current values. An enzyme-coated electrode using a metal-enzyme hybrid can greatly improve the performance of the biosensor, and allow it to be used in highly complex systems, such as, for example, enzyme-channeling based immunosensors.

Integration of biologically active proteins in nano-electric circuitry or magnetically-based devices requires the acquisition of electric conductivity and/or magnetism to these proteins, which are typically devoid of such properties, without sacrificing their native structure and properties, as well as the biological activity which stems therefrom. One technique which can be used to partially or fully plate a protein with a metal coat is the electrochemical technique known as electroless deposition.

Electroless deposition is a widely known technique for depositing metals, such as magnetic and/or conductive metals, on a variety of surfaces including biologically active surfaces. This technique is widely used in the electronics industry to manufacture conductors, semiconductors and other elements which require a metal finish by plating nickel, cobalt, palladium, platinum, copper, gold, silver and other metals and alloys thereof. Electroless deposition is presently known as a highly suitable technique for forming metal films and coatings on microscopic elements and areas on substrates surfaces, for forming barriers and interconnects between different layers on semiconducting wafers and for creating microscopic reservoirs of metallic atoms at specific sites of a subject carrier element. Hence, at present, electroless deposition is mostly utilized in the manufacture of devices on semiconductor wafers, and particularly in the fabrication of multiple levels of conductive layers on a substrate surface.

In principle, electroless deposition is performed in electrolytic solutions or fluids (e.g., aqueous solutions of metal ions) without applying an external voltage, and is effected by an electrochemical reaction between the metal ions and a reducing agent. The electrolytic solution may optionally further include complexing agents and pH adjusting agents and the process can optionally be performed on a catalytic surface (e.g., of a semiconductor wafer).

Apart of its simplicity, electroless deposition offers other advantages over other metal plating techniques such as, for example, electro-deposition, chemical vapor deposition and high-vacuum sputtering. These advantages include smooth and uniform (“bumpless”) coverage of large, uneven and complex surfaces, plating under non-aggressive or corrosive conditions, plating of non-conductive surfaces, and the absence of an electric current in the process.

Electroless deposition has been used to plate, for example, lipid- and peptide-based tubular structures and self-assembled monolayers with various metals, and it is further presently utilized in several biological and medical applications. One example for such an application is the treatment and prevention of tooth cavities, which is effected by depositing a thin metal film onto tooth enamel. The deposited metal films exhibited high adherence to the tooth and maintained the bulk metal properties.

Other examples include metallization of various biological moieties by electroless deposition. Thus, electroless deposition of natural arrays of proteins was recently successfully demonstrated for the fabrication of nanowires from microtubules, viral envelopes, amyloid fibers and actin filaments.

The metallization of the biological moieties described above was effected by techniques that involve nucleation and enlargement by electroless plating. Nucleation was typically performed by adsorption of palladium or platinum ions onto the surface of the biological moiety, followed by chemical reduction thereof, or, alternatively, by surface labeling with colloidal gold particles. Enlargement of the nucleation sites thus obtained into continuously deposited metallic films was typically carried out by immersion in a plating solution containing the metal ions of choice (e.g., Ag⁺¹ or Ni⁺²) and reducing agents (e.g., NaBH₄ or dimethylaminoborane). These techniques typically result in the formation of a relatively thick metal deposition, of e.g., 10 to 35 nanometers [Y. Yang et al., J. Mater. Sci. 2004, 39, 1927-1933]. These techniques further lead to the loss of the proteins native biological activity due to deformation and denaturation, blockage of active and binding sites, and gross precipitation of the protein, which most likely results from the strong and incontrollable reducing aptitude of the reducing agent used.

Thus, while the presently known methods for metallizing biological moieties by electroless deposition involve proteins that are either immobilized and/or inactivated before, during and/or as a result of the deposition process, the ability and utility to deposit metals onto a single, soluble biological moiety, particularly protein, while maintaining its activity, dissolvability and other parameters has not been demonstrated hitherto. Such a metallization should be performed while maintaining features such as the native chemical structure, the motility and thus the biological activity of the protein. The presently known electroless deposition methods, however, typically interfere with these features and hence do not allow the provision of metallized yet active proteins. As discussed hereinabove, metal deposition onto a biological entity, in a molecular level, is highly desired in various applications and particularly in the field of nanowiring.

European Patent No. EP00173629B1 teaches the attachment of metal-ion chelating moieties to the surface of antibodies, to thereby form conjugates of antibodies and chelating moieties while maintaining the immunoreactivity and immunospecificity of the antibodies towards their corresponding antigen. The attachment of the chelating moieties, according to this patent, is effected by generation of aldehyde groups on the surface glycans of the antibody by oxidation, followed by the conjugation thereto of chelating moieties that have a free amine group, so as to form, under mild conditions, a Schiff-base between the aldehyde group on the antibody's surface and the amine group of the chelating moiety. Alternatively according to this patent, the attachment of the chelating moieties is effected by generation of sulfhydryl groups on the surface of the antibody by reduction of disulfide groups, followed by the conjugation thereto of chelating moieties that have certain reactive groups capable of reacting with a sulfhydryl, so as to form a bond between the sulfhydryl group on the antibody's surface and the chelating moiety. The resulting conjugate is then used for complexing discrete metal ions via the chelating moieties. This patent is directed mainly at complexing discrete ions of radioisotopes to antibodies which can then be used in various nuclear medicine practices. This patent, however, is completely silent with respect to the deposition of continuous patches of elemental metal, or an alloy, comprising a plurality of contiguous atoms on the surface of a protein, hence this patent fails to teach or suggest the electroless metal deposition on proteins, while maintaining the activity or dissolvability of the proteins.

PCT/IL2006/000115 by Freeman et al., a co-inventor of the present invention, teaches methods of electroless deposition of silver on proteins surface, while maintaining the activity and/or dissolvability of the proteins. The metal-coated proteins according to PCT IL2006/000115 are prepared by selectively modifying portions of the protein surface so as to attach reducing moieties thereto, such as imine, hydrazine and hydrazide groups, whereby these reducing moieties participates in an effective, yet controllable in-situ electroless deposition of continuous amorphous and/or crystalline silver patches onto the proteins surface, to thereby form the silver-coated proteins. PCT/IL2006/000115 further teaches silver-coated glucose oxidase-containing biosensors for detecting glucose in a liquid sample. According to PCT/IL2006/000115, the deposition of metallic silver on the surface of the protein is directly effected by contacting the reducing moieties-containing protein surface with silver ions and is enabled by the low redox potential of the silver and the high reduction aptitude of the reducing moieties, which allows performing the deposition under conditions which do not affect the protein's characteristics. The metallic silver deposition can be performed in a site-specific manner, by pre-selecting that portion of the protein surface that is subjected to modification (by attaching thereto the reducing moieties).

Yet, novel and general processes for electroless deposition of thin layers of metals other than silver, such as palladium, cobalt, nickel and copper, on the surface of proteins, as well as particles and cells which comprise proteins, while maintaining their innate biological activity are highly desirable.

There is thus a widely recognized need for, and it would be highly advantageous to have a novel method for depositing thin layers of metals on the surface of proteins such as enzymes and cell surfaces, which would allow the preparation of metal-coated yet dissolvable and active proteins.

### SUMMARY OF THE INVENTION

According to one aspect of the present invention there is provided a composition-of-matter comprising a protein having a surface and a metal coating deposited over at least a portion of the surface and forming a metal-coated protein being dissolvable or suspendable in an aqueous medium, the metal being selected from the group consisting of a single metal and a combination of at least two metals, the single metal being devoid of silver.

According to still further features in preferred embodiments of the invention described below, the protein has a biological activity and the metal-coated protein retains the biological activity.

According to another aspect of the present invention there is provided a composition-of-matter comprising a protein having a surface and further having a biological activity and a metal coating deposited over at least a portion of the surface and forming a metal-coated protein retaining the biological activity, the metal being selected from the group consisting of a single metal and a combination of at least two metals, the single metal being devoid of silver.

According to still further features in preferred embodiments of the invention described below, the metal-coated protein is dissolvable or suspendable in an aqueous medium.

According to still another aspect of the present invention there is provided a composition-of-matter comprising a protein having a modified surface and a metal coating deposited over at least a portion of the surface and forming a metal-coated protein, the modified surface having at least one chelating moiety attached thereto, the chelating moiety being for forming a complex with ions of the metal.

According to still further features in preferred embodiments of the invention described below, the protein has a biological activity and the metal-coated protein retains the biological activity.

According to still further features in the described preferred embodiments the metal-coated protein is dissolvable or suspendable in an aqueous medium.

According to yet another aspect of the present invention there is provided a composition-of-matter comprising a protein having a modified surface and a plurality of ions of a metal attached to at least a portion of the surface, the modified surface having a plurality of chelating moieties attached thereto and the chelating moieties being for forming a complex with the ions of the metal.

According to still further features in preferred embodiments of the invention described below, the metal-coated protein is prepared by contacting a modified protein having at least one chelating moiety attached to the surface with a reducing agent, the chelating moiety being for forming a complex with ions of the metal.

According to an additional aspect of the present invention there is provided a process of preparing a metal-coated protein, the process comprising: reacting the protein with at least one chelating moiety, to thereby obtain a modified protein having the chelating moiety attached to at least a portion of a surface thereof, the chelating moiety being for forming a complex with ions of the metal, contacting the modified protein with a first aqueous solution containing ions of the metal to thereby obtain a solution containing a complex of the modified protein and the metal ions; and contacting the solution containing the complex of the modified protein and the metal ions with a first reducing agent, the first reducing agent being for reducing the ions of the metal, thereby obtaining the metal-coated protein.

According to still further features in preferred embodiments of the invention described below, the process further comprises, subsequent to or concomitant with the contacting with the first reducing agent: contacting the metal-coated protein or the solution containing the complex, with a second aqueous solution containing a plurality of ions of a second metal, in the presence of a second reducing agent, the second reducing agent being for reducing the ions of the second metal, to thereby obtain the metal-coated protein having an additional coating of the second metal on the surface.

According to still further features in the described preferred embodiments reacting the protein with the at least one chelating moiety comprises: modifying at least a portion of a surface of the protein, to thereby obtain a modified protein having a plurality of reactive groups on the surface; and conjugating to at least a portion of the reactive groups the chelating moiety.

According to still an additional aspect of the present invention there is provided a pharmaceutical composition comprising, as an active ingredient, the composition-of-matter described hereinabove and a pharmaceutically acceptable carrier.

According to still further features in preferred embodiments of the invention described below, the pharmaceutical composition is packaged in a packaging material and identified in print, in or on the packaging material, for use in the treatment of a bacterial and/or fungal infection.

According to yet an additional aspect of the present invention there is provided a method of treating a bacterial and/or fungal infection, the method comprising administering to a subject in need thereof a therapeutically effective amount of the composition-of-matter described herein.

According to a further aspect of the present invention there is provided a use of the composition-of-matter described herein in the preparation of a medicament. The medicament being preferably for the treatment of a bacterial and/or fungal infection.

According to still a further aspect of the present invention there is provided a metallic element comprising the composition-of-matter described herein.

According to yet a further aspect of the present invention there is provided an electronic circuit assembly comprising an arrangement of conductive elements interconnecting a plurality of electronic elements wherein at least a portion of the conductive elements comprises the metallic element described herein.

According to another aspect of the present invention there is provided a device comprising a plurality of the metallic elements described herein.

According to a further aspect of the present invention there are provided an electrode comprising the composition-of-matter described herein deposited thereon.

According to still a further aspect of the present invention there is provided a biosensor system for electrochemically determining a level of an analyte in a liquid sample, the system comprising: an insulating base; and an electrode system which comprises the electrode described hereinabove, wherein the protein is selected capable of chemically reacting with the analyte while producing a transfer of electrons.

According to still a further aspect of the present invention there is provided method of electrochemically determining a level of an analyte in a liquid sample, the method comprising: contacting the biosensor system of claim 60 with the liquid sample; and measuring the transfer of electrons, thereby determining the level of the analyte in the sample.

According to an additional aspect of the present invention there is provide an imaging probe comprising the composition-of-matter described herein, wherein the metal in the metal-coated protein comprises a detectable metal.

The present invention successfully addressed the shortcomings of the presently known configurations by providing a novel methodology for depositing a metal coat on a protein surface while substantially maintaining the activity and dissolvability of the protein.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

As used herein, the singular form “a”, “an” and “the” include plural references unless the context clearly dictates otherwise. For example, the term “a protein” or “at least one protein” may include a plurality of proteins, including mixtures thereof.

Throughout this disclosure, various aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein throughout, the term “comprising” means that other steps and ingredients that do not affect the final result can be added. This term encompasses the terms “consisting of” and “consisting essentially of”.

The term “method” or “process” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a schematic illustration of an enzyme/palladium ion complex, according to preferred embodiments of the present invention, showing an enzyme (blob-shaped object) surface-modified by PGA chains (tilde-shaped lines), to which a plurality of chelating moieties are attached (C-shaped crescents), complexing Pd²⁺ ions (dots);

FIG. 2 presents comparative plots demonstrating the reduction rate of palladium atoms, detected as a change in optical density measured at 322 nm as a function of palladium ion concentration and time, showing no change in the optical density (O.D.) for a sample of an enzyme/palladium ion complex prepared with 2 mM Pd²⁺ without a reducing agent (blue diamonds, denoted “GOX-PGA-IDA-Pd⁺⁺ (2 mM) No HP”), no change in O.D. for a sample of an enzyme/palladium ion complex prepared with 0.5 mM Pd²⁺ in the presence of a reducing agent (cyan crosses, denoted “GOX-PGA-IDA-Pd⁺⁺ (0.5 mM)+HP”), no change in O.D. for a sample of an enzyme/palladium ion complex prepared with 1 mM Pd²⁺ in the presence of a reducing agent (yellow triangle, denoted “GOX-PGA-IDA-Pd⁺⁺ (1 mM)+HP”), and a gradual increase in O.D. for a sample of an enzyme/palladium ion complex prepared with 2 mM Pd²⁺ in the presence of a reducing agent (magenta squares, denoted “GOX-PGA-IDA-Pd⁺⁺ (2 mM)+HP”);

FIG. 3 presents comparative plots demonstrating the reduction and deposition rate of additional palladium atoms detected as a change in optical density measured at 322 nm as a function of time, in a sample of an enzyme/palladium ion complex without a reducing agent (blue diamonds, denoted “GOX-PGA-IDA-Pd⁺⁺ (No HP)”), in a sample of an enzyme/palladium ion complex in the presence of a reducing agent (yellow triangles, denoted “GOX-PGA-IDA-Pd⁺⁺+HP”), and in a sample of an enzyme/palladium ion complex in the presence of a reducing agent and additional palladium ions, (magenta circles, denoted “GOX-PGA-IDA-Pd⁺⁺+HP+Pr⁺⁺”);

FIG. 4 presents comparative plots demonstrating the reduction and deposition rate of additional palladium atoms detected as a change in optical density measured at 322 nm as a function of time, in a sample of an enzyme/palladium ion complex contacted with a reducing agent without additional Pd²⁺ ions (blue diamonds, denoted “GOX-PGA-IDA-Pd⁺⁺+HP”), a sample of an enzyme/palladium ion complex contacted with a reducing agent and a solution of 0.5 mM Pd²⁺ ions (magenta squares, denoted “GOX-PGA-IDA-Pd⁺⁺+HP+Pd⁺⁺0.5 mM”), a sample of an enzyme/palladium ion complex contacted with a reducing agent and a solution of 1 mM Pd²⁺ ions (yellow triangles, denoted “GOX-PGA-IDA-Pd⁺⁺+HP+Pd⁺⁺1 mM”), and a sample of an enzyme/palladium ion complex contacted with a reducing agent and a solution of 2 mM Pd²⁺ ions (cyan exes,denoted “GOX-PGA-IDA-Pd⁺⁺+HP+Pd⁺⁺2 mM”);

FIG. 5 presents a high resolution electron micrograph, obtained without further staining of the sample, of a layer of palladium atoms deposited on the surface of glucose oxidase according to preferred embodiments of the present invention, by modifying the enzyme's surface with polyglutaraldehyde and iminodiacetate, and complexing thereto palladium ions, and further by reducing the ions with hypophosphite (HP) with further addition of palladium ions, showing a patch of about 10 nm in diameter of crystalline palladium on the surface of the enzyme (scale bar of 2 nm);

FIG. 6 presents an electron dispersion spectroscopy (EDS) spectrograph of a patch of palladium deposited on glucose oxidase according to preferred embodiments of the present invention as shown in FIG. 5, demonstrating the presence of palladium in the patch, and showing peaks of carbon and oxygen stemming from the protein, peaks of phosphorous stemming from the reducing agent and peaks for copper stemming from the sample microgrid;

FIGS. 7A-F present high resolution electron micrographs, obtained without further staining of the sample, of patches of copper (FIGS. 7A and 7B), cobalt (FIGS. 7C and 7D) and nickel (FIGS. 7E and 7F), deposited on the surface of glucose oxidase according to preferred embodiments of the present invention, by modifying the enzyme's surface with polyglutaraldehyde and iminodiacetate, and complexing thereto palladium ions, and further by reducing the palladium ions with hypophosphite (HP) and contacting the resulting palladium-coated enzyme with a solution of copper ions (FIGS. 7A and 7B), cobalt ions (FIGS. 7C and 7D) and nickel ions (FIGS. 7E and 7F), showing round patches ranging from about 5 nm to about 20 nm in diameter of amorphous and crystalline metal on the surface of the enzyme (scale bar for FIGS. 7A, 7E and 7F is 5 nm, scale bar for FIGS. 7B and 7C is 2 nm, and scale bar for FIG. 7D is 10 nm);

FIG. 8 presents images of five transparent test-tubes serving in a visual dissolvability assay, showing a clear sample of unmodified glucose oxidase and no palladium ions, denoted “GOX—untreated”; a clear and substantially untinted sample of glucose oxidase modified with polyglutaraldehyde and iminodiacetate and complexed palladium ions, denoted “GOX-PGA-IDA-Pd⁺⁺No HP”; a sample of palladium ions reduced by hypophosphite without an enzyme having a precipitation of insoluble metallic particles at the bottom of the test-tube, denoted “Pd⁺⁺+HP (no GOX)”; a lightly tinted yet clear (soluble) sample of an enzyme/metallic palladium complex, denoted “GOX-PGA-IDA-Pd⁺⁺+HP”, and a darkly tinted yet clear (soluble) sample of an enzyme/metallic palladium complex having a thickened layer of metallic palladium deposited on the surface of the enzyme, denoted “GOX-PGA-IDA-Pd⁺⁺+HP+Pd⁺⁺”;

FIGS. 9A-B present comparative cyclic voltammograms of electro-catalytic currents (in microamperes) plotted versus electric potential (in millivolts) recorded in five reiterations for a sample of native glucose-oxidase (FIG. 9A), and in six reiterations for a sample of cobalt-coated glucose-oxidase (FIG. 9B), an exemplary metal-coated protein according to preferred embodiments of the present invention, showing an improved electric current response in the cobalt-coated enzyme; and

FIG. 10 presents comparative chronoamperometric plots recorded for a modified working electrode having deposited thereon untreated glucose oxidase (blue line), polyglutaraldehyde-treated glucose oxidase (green line), PGA and IDA-treated glucose oxidase (red line), and PGA and IDA-treated glucose oxidase coated with palladium (black line).

### DESCRIPTION OF THE PREFERRED EMBODIMENTS

The present invention is of metal-coated proteins, which substantially retain the biological activity and/or dissolvability of the corresponding native (uncoated) protein and can therefore be utilized in various applications such as, for example, therapeutic applications and in forming electronic devices. The metal-coated proteins according to the present invention are prepared by contacting a modified protein having metal ions complexed with chelating moieties that are attached to the surface thereof with a relatively mild reducing agent, so as to effect an effective, yet controllable in-situ electroless deposition of the metal onto the proteins surface. The present invention is therefore further of such modified proteins and of methods of preparing the metal-coated proteins. The modification of the protein surface and the reduction are performed under mild conditions that do not affect the protein structural and chemical properties. The present invention is further of pharmaceutical compositions containing and methods of treating infections utilizing biologically active (biocidal) metal-coated proteins. The present invention is further of metallic elements comprised of the metal-coated proteins, and of electronic circuits, devices and an imaging probes containing same. The present invention is further of electrodes having the metal-coated proteins deposited thereon, of biosensors containing same and of uses thereof for electrochemically detecting analytes, such as glucose in liquid samples.

The principles and operation of the present invention may be better understood with reference to the figures and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

As discussed hereinabove, and further discussed in PCT/IL2006/000115 (supra), electroless deposition is a highly beneficial technique for depositing metals on various sensitive surfaces, such as proteins. Since native proteins typically do not promote metal deposition onto their surface, a novel methodology for performing Electroless deposition has been sought.

While conceiving the present invention, it was envisioned that attaching metal ions onto the surface of the protein, and thereafter reducing these ions in-situ, using a mild reducing agent, while retaining the protein's biological activity, dissolvability and other functionally essential features, would form a coat of elemental (zero valence) metal atoms on the surface of the protein.

It was further envisioned that in order for the coating process to take place as desired, several key criteria must be maintained: the modification of the protein surface so as to enable attaching thereto metal ions as well as the reduction process must be effected and use reagents which would be mild so as not to compromise the biological activity and dissolvability of the protein, and the metal ions must have a reduction potential that would enable to perform the reduction process under these reduction conditions while being directly attached to the surface of the protein.

While further conceiving the present invention, the present inventors have devised a methodology for attaching a metal-ion chelating functionality to the surface of a protein, to thereby provide the means for attaching a plurality of such ions to the protein. This methodology calls for utilizing naturally occurring functional groups on the protein for attaching thereto multifunctional substances that have a plurality of reactive groups, and thereafter conjugating chelating moieties to these reactive groups. Using such modified proteins, metal ions could be attached to the protein surface by complexation and could be reduced in-situ in the presence of a mild reducing agent so as to form a metal coat on the protein's surface.

While reducing the present invention to practice the present inventors have successfully modified a protein so as to have polyglutaraldehyde attached to the amine groups of naturally occurring lysine residues on the protein's surface, and further successfully conjugated chelating moieties to the thus generated free aldehyde groups on the protein surface. The resulting chelating moieties-containing modified protein was shown to form a complex with metal cations in solution, and the thus obtained metal-protein complex were further successfully subjected to in-situ reduction, using a mild reducing agent, which resulted in formation of elemental metal atoms onto the protein surface, thereby achieving the formation of a metal coat on the surface of the protein while substantially maintaining it dissolvability and biochemical activity, as demonstrated in the Examples section that follows.

Hence, according to one aspect of the present invention, there is provided a composition-of-matter which comprises a protein having a surface and further characterized by its innate biological activity and dissolvability, and a metal coating deposited over at least a portion of its surface, thus forming a metal-coated protein. The metal-coated protein is substantially dissolvable and/or suspendable in aqueous solutions which are typically suitable for dissolving proteins, and/or further substantially retains its original characteristic biological activity. According to this aspect of the present invention, the metal coat may consist of a single metal or a combination of two or more metals, whereby in case that a single metal is used for the metal coat, it can be any metal other than silver.

As used herein, the phrase “substantially retaining”, which is also referred to herein interchangeably as “substantially maintaining” and used with respect to the protein's properties, refers to protein's properties such as specific activity, dissolvability and other biochemical properties essential to its biological activity, which are retained or maintained at significant levels subsequent to the chemical modifications described herein. A “significant level” in this respect refers to at least 10% of the corresponding property of a corresponding native protein, preferably at least 20%, more preferably at least 30%, more preferably at least 40% and more preferably at least 50% and even at least 70%, 80% and up to 100% of the corresponding property of a corresponding native (uncoated) protein.

Herein, the terms “dissolvable” or “suspendable” and their synonymous term “soluble” are used to describe the capability of a single protein molecule to be dissolved or suspended in an aqueous solution or media.

As discussed hereinabove, the metal-coated proteins presented herein can be prepared by contacting a modified protein having one or more chelating moieties attached to its surface with a reducing agent, as this phrase is defined hereinbelow, whereby the chelating moiety are selected capable of forming a complex with ions of the metal.

Thus, according to another aspect of the present invention there is provided a composition-of-matter, which comprises a protein having a modified surface and a metal coating deposited over at least a portion of the surface and forming a metal-coated protein, wherein the modified surface has one or more chelating moieties attached thereto, for forming a complex, such as an organometallic complex, with ions of the metal(s), as defined and discussed in detail hereinbelow.

The phrase “modified protein” as used to herein, describes a protein that has been subjected to a chemical modification and, specifically, to modification of at least some of its surface groups. In the context of the present invention, the chemical modification results in conjugation of a chelating moiety to the protein surface and hence, unless otherwise indicate, this phrase is used herein to describe a protein that has one or more chelating moieties conjugated to its surface.

In any of the aspects of the present invention described herein, the utilized protein can be any naturally occurring, synthetic or synthetically modified protein including, but not limited to, an antibody (including fragments thereof), a lectin, a glycoprotein, a lipoprotein, a nucleic acid binding protein, a cellular protein, a cell surface protein, a viral coat (capsid) protein, a serum protein, a growth factor, a hormone, an enzyme and a transcription factor, all are characterized by a specific biological activity.

It is assumed that in some cases, other types of proteins, which in their native form are attached to an insoluble matrix, such as a membrane, or otherwise immobilized, can be partially coated with metal according to some aspects of the present invention, and still maintain their biological activity. Such proteins may include proteins of the intra- and extra-cellular matrices, membranal proteins such as receptors and channels, fibrous proteins, viral-coat proteins and fragments thereof.

Thus, the protein utilized in the context of the present embodiments can be a protein that forms a part of a cell (a cellular protein). An example of a cellular protein is a cell-surface protein, or a membrane protein. Metallization of such proteins can practically result in metal-coating the cell either partially or entirely, depending on the density of the protein on the surface of the cell. The same concept applies to single cells as to cells which form a part of a multi-cell organism, a tissue or an organ. The same concept applies to viral coat proteins, via which a virus can be completely or partially coated with a metal.

According to a preferred embodiment of the present invention, the protein is an enzyme and the composition-of-matter comprises a metal-coated enzyme, which is characterized by being dissolvable in an aqueous medium, and by retaining its specific biological catalytic activity.

As is demonstrated in Examples section that follows (see, Example 3), a palladium-, nickel-, cobalt- and/or copper-coated enzyme and, more specifically, a palladium-, nickel-, cobalt- and/or copper-coated glucose oxidase, was successfully prepared using the methodologies described herein. The metal-coated enzyme was assayed for its residual specific activity and dissolvability after each step of the process and was shown to retain a significant level of these characteristics, as compared with its activity prior to any chemical modification, as these are described hereinbelow, and after the deposition of the metal(s) coat on at least a portion of its surface.

Hence, according to a preferred embodiment of the present invention, the protein, onto which a metal coat is applied, is the enzyme glucose oxidase. For general information regarding this enzyme, see the Examples section that follows.

The metal coat may comprise a single metal element, or a combination of two or more metal elements. When more than one metal is deposited on the protein surface, the two or more metals can be deposited simultaneously, so as to form a coat layer that comprises a combination of these metals (as in an alloy), or, preferably, one metal is first deposited on the protein surface and may form nucleation sites, whereby the other metals are deposited thereon, so as to form a doubly-layered or multi-layered metal coat or gain, an alloy.

Each of the metals forming the metal coat can be, for example, a conductive metal, a semi-conductive metal, a magnetic metal, and/or a radioactive metal isotope, and hence can be selected upon the intended use of the composition-of-matter comprising the metal-coated protein.

Preferably, the metal is a transition metal, a rare-earth metal and any alloy or mixture thereof.

Representative examples of metals that are suitable for use in this context of the present invention include, without limitation, palladium, copper, gold, chromium, nickel, cobalt, iron, cadmium, platinum, silver, uranium, iridium, zinc, manganese, vanadium, rhodium, ruthenium, mercury, arsenic, antimony, and any combination thereof. Preferably the metal is any one of palladium, copper, nickel, cobalt or a combination thereof.

In general, a metal is selected such that it has a reduction potential that is compatible with the selected reducing agent, whereby both the reducing agent and the metal are selected such that the reduction process, which is performed in the vicinity of the protein surface, could be performed under physiological conditions (aqueous solutions and a temperature not higher than 40° C.).

In a preferred embodiment, the metal is palladium. As discussed in detail hereinbelow, elemental palladium is known as forming efficient nucleation sites. Hence, palladium atoms deposited on a protein surface can form nucleation sites for additional deposition of other metals, and particularly of metals that possess the desired characteristic for a certain application, as is detailed hereinbelow. Thus, for example, the additional metal can be palladium itself, a magnetic metal such cobalt, a semi-conductive metal such as copper or nickel, a radioactive metal and so forth.

The deposited metal coat on the surface of the protein covers at least a portion of the protein surface. As used herein, the term “at least a portion” describes a certain portion of the protein, which is determined as described hereinabove. This portion can range from about 0.01% of the protein surface to substantially all the protein surface.

According to a preferred embodiment of the present invention, the metal-coat on the surface of the protein covers from about 0.1% to about 90% of the solvent-accessible surface of the protein.

The metal coat can be either in the form of a continuous metallic layer, covering parts or all of the surface, or in the form of one or more separate metal particles deposited on one or more sites of the protein surface.

Depending, at least in part, on the metal type, the reducing agent used and the rate of the reduction process, the deposited metal may be in a crystalline form, having a well-ordered structure. Alternatively, the deposited metal can be in an amorphous form or deposited as a mixture of both morphologies, namely crystalline and amorphous. Preferably, the deposited metal has a crystalline form, which is highly suitable, for example, for applications where electronic conductivity, magnetism and/or spectral properties are desired.

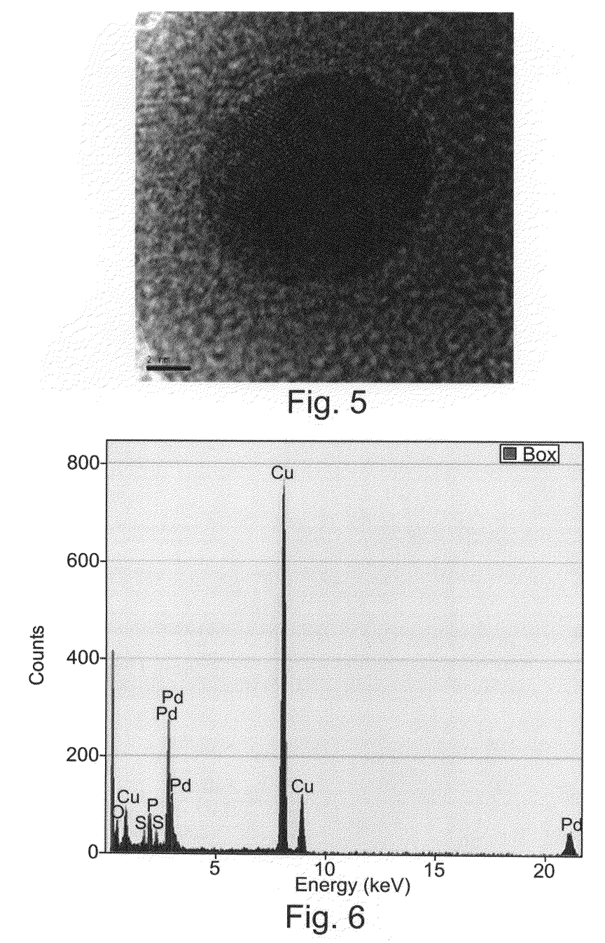

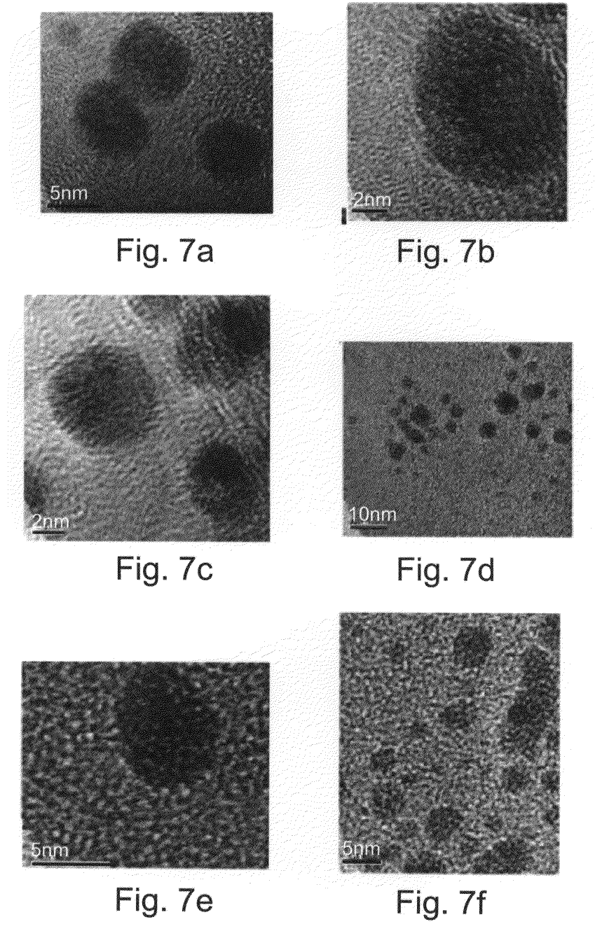

Regardless of its form, preferably, the metal coat is a nano-sized coat. Thus, when the metal coat has a form of a continuous layer, preferably, the layer's thickness ranges from about 0.1 nanometer to about 10 nanometers. When the metal coat has a form of particles, preferably, the size of a single deposited metal particle ranges from about 1 nanometer to about 100 nanometers in diameter, more preferably from about 1 nanometer to about 50 nanometers. Micrographs of a portion of an exemplary palladium-coated protein, prepared according to the methodology described herein, are presented in FIGS. 5 and 7, and show patches of about 5 nm to about 20 nm in diameter of crystalline and semi-crystalline metals deposited on the surface of a protein.

According to preferred embodiments, the molar ratio between the protein and the metal in the composition-of-matter presented herein ranges from about 1:10 protein to about 1:10000 moles protein to moles metal, preferably from about 1:100 to about 1:1000.

As discussed hereinabove, the metal-coated protein presented herein is a modified protein having chelating moieties attached to its surface. These chelating moieties serve for forming a metal ion-protein complex between metal ions and these chelating moieties, prior to reducing the metal ions so as to form the metal-coated protein.

As used herein, the phrase “chelating moiety” describes a chemical moiety that is capable of forming a stable complex, such as an organometallic complex, with a metal, typically by donating electrons from certain electron-rich atoms present in the moiety to an electron-poor metal.

Chelating moieties typically contain one or more chelating groups. The phrase “metal-coordinating group”, also referred to herein and in the art as a “dentate”, describes that chemical group in the chelating moiety that contains a donor atom. The phrase “donor atom” describes en electron-rich atom that can donate a pair of electrons to the coordination sphere of the metal. Typical donor atoms include, for example, nitrogen, oxygen, sulfur and phosphor, each donating two (lone pair) electrons.

Representative examples of metal-coordinating groups that may be included in the chelating moieties according to the present embodiments therefore include, without limitation, amine, imine, carboxylate, beta-ketoenolate, thiocarboxyl, carbonyl, thiocarbonyl, hydroxyl, thiohydroxyl, hydrazine, oxime, phosphate, phosphite, phosphine, alkenyl, alkynyl, aryl, heteroaryl, nitrile, azide, alkoxy and sulfoxide.

As used herein, the term “amine” refers to an —NR′R″ group where R′ and R″ are each hydrogen, alkyl, alkenyl, cycloalkyl, aryl, heteroaryl (bonded through a ring carbon) or heteroalicyclic (bonded through a ring carbon) as defined hereinbelow.

The term “alkyl” as used herein, describes a saturated, substituted or unsubstituted aliphatic hydrocarbon including straight chain and branched chain groups. Preferably, the alkyl group has 1 to 20 carbon atoms. Whenever a numerical range; e.g., “1-20”, is stated herein, it implies that the group, in this case the alkyl group, may contain 1 carbon atom, 2 carbon atoms, 3 carbon atoms, etc., up to and including 20 carbon atoms. More preferably, the alkyl is a medium size alkyl having 1 to 10 carbon atoms. Most preferably, unless otherwise indicated, the alkyl is a lower alkyl having 1 to 5 carbon atoms.

The term “alkenyl” refers to an alkyl group, as defined herein, which consists of at least two carbon atoms and at least one carbon-carbon double bond.

The term “cycloalkyl” describes an all-carbon, substituted or unsubstituted monocyclic or fused ring (i.e., rings which share an adjacent pair of carbon atoms) group where one or more of the rings does not have a completely conjugated pi-electron system.

The term “heteroalicyclic” describes a substituted or unsubstituted monocyclic or fused ring group having in the ring(s) one or more atoms such as nitrogen, oxygen and sulfur. The rings may also have one or more double bonds. However, the rings do not have a completely conjugated pi-electron system.

The term “aryl” describes an all-carbon monocyclic or fused-ring polycyclic (i.e., rings which share adjacent pairs of carbon atoms) groups having a completely conjugated pi-electron system.

The term “heteroaryl” describes a substituted or unsubstituted monocyclic or fused ring (i.e., rings which share an adjacent pair of atoms) group having in the ring(s) one or more atoms, such as, for example, nitrogen, oxygen and sulfur and, in addition, having a completely conjugated pi-electron system. Examples, without limitation, of heteroaryl groups include pyrrole, furane, thiophene, imidazole, oxazole, thiazole, pyrazole, pyridine, pyrimidine, quinoline, isoquinoline and purine.

As used herein, the term “carboxylate” refers to an —(═O)OR′ group, where R′ is as defined herein.

As used herein, the term “beat-ketoenolate” refers to a —R—C(═O)—CR′R″—C(═O)—R′″ group, where R′ and R″ are as defined herein, R and R′″ are as defined herein for R′ and R″.

The term “imine”, which is also referred to herein and in the art interchangeably as “Schiff-base”, describes a —N═CR′— group, with R′ as defined herein. As is well known in the art, Schiff bases are typically formed by reacting an aldehyde and an amine-containing moiety such as amine, hydrazine, hydrazide and the like, as these terms are defined herein.

As used herein, the term “thiocarboxylate” refers to an —C(═S)OR′ group, where R′ is as defined herein.

As used herein, the terms “carbonyl” as well as “acyl” refer to a —C(═O)-alkyl group, as defined hereinabove.

The term “thiocarbonyl” as used herein, describes a —C(═S)—R′, with R′ as defined herein.

The term “hydroxyl” describes a —OH group.

As used herein, the term “thiol” or “thiohydroxy” refers to a —SH group.

The term “phosphate” describes a —O—P(═O)(OR′)(OR″) group, with R′ and R″ as defined herein.

The term “phosphite” describes an —O—PR′(═O)(OR″) end group or an PR′(═O)(O)— linking group, as these phrases are defined hereinabove, with R′ and R″ as defined herein.

The term “phosphine” describes a —PR′R″R′″ group, with R′, R″ and R′″ as defined herein.

The term “oxime” describes a ═N—OH group.

The term “nitrile” or “cyano” describes a —C≡N group.

The term “isocyanate” describes a —N═C═O group.

The term “azide” describes a —N₃ group.

The term “alkoxy” as used herein describes an —O-alkyl, an —O-cycloalkyl, as defined hereinabove.

As used herein, the term “thioalkoxy” describes both a —S-alkyl, and a —S—cycloalkyl, as defined hereinabove.

The term “sulfoxide” or “sulfinyl” describes a —S(═O)R′ end group or an —S(═O) linking group, as these phrases are defined hereinabove, where R′ is as defined hereinabove.

A chelating moiety, according to preferred embodiments, can be a monodentate chelating moiety, having one metal-coordinating group, a bidentate chelating moiety having two metal-coordinating groups, a tridentate chelating moiety having three metal-coordinating groups, a tetradentate chelating moiety having four metal-coordinating groups, or a chelating moiety having more than four metal-coordinating groups.

Thus, for example, the phrase “bidentate chelating moiety”, as used herein, describes a chelating moiety that contains two metal-coordinating groups linked one to the other (and hence provides two donor atoms), as described hereinabove, and thus can coordinatively bind two coordination sites of the metal. Representative examples of bidentate chelating moieties include, without limitation, ethylenediamine, 2-mercapto-ethanol, 2-amino-ethanethiol, 3-amino-propan-1-ol, 2-amino-3-mercapto-propionic acid (cysteine), acetylacetonate and phenanthroline.

The chelating moiety is selected suitable for forming a stable complex with the desired metal. The stability of the metal-coordination complex typically depends on the number, type and spatial arrangement of the metal-coordinating groups surrounding the metal ion(s) and their fit to the coordination sphere of the metal.

Thus, for example, metals such as cadmium, chromium, cobalt, copper, gold, iridium, iron, lead, magnesium, manganese, mercury, nickel, palladium, platinum, rhodium, ruthenium, silver, vanadium and/or zinc are known to form stable complexes with metal-coordination groups such as, for example, amine, imine, carboxylate, carbonyl, phosphine, nitrile and hydroxyl. Thus, for forming proteins having one or more of these metals deposited thereon, modified proteins having chelating moieties that include one or more of these metal-coordinating groups are preferably utilized. Examples of chelating moieties having such metal-coordinating groups and which can preferably be utilized to complex these metals include, without limitation, iminodiacetate, ethylenediamine, diaminobutane, diethylenetriamine, triethylenetetraamine, bis(2-diphenylphosphmethyl)amine, and tris(2-diphenylphosphmethyl)amine.

Similarly, metals such as mercury, arsenic, antimony and gold, are known to form stable complexes with metal-coordination groups such as amine, thiohydroxyl, hydroxyl, thiocarboxyl thiocarboxylate, thioalkoxy, thiosemicarbazide and thiocarbonyl. Thus, for forming proteins having one or more of these metals deposited thereon, modified proteins having chelating moieties that include one or more of these metal-coordinating groups are preferably utilized. Examples of chelating moieties having such metal-coordinating groups and which can preferably chelate these metals include, without limitation, dimercaprol, 2-mercapto-ethanol, 2-amino-ethanethiol, 3-amino-propan-1-ol, 2-amino-3-mercapto-propionic acid (cysteine), amidomercaptoacetyl, acetylacetonate and phenanthroline.

Correspondingly, transition metals such as techtenium and/or rhenium, optionally and preferably in the oxidized forms thereof oxorhenium(V) and oxotechnetium(V), are known to form stable complexes with metal-coordination groups such amine, oxime, hydrazine and thiol. Preferably these metals require a four metal-coordinating groups for optimal coordination, hence, preferred complexes of oxorhenium(V) and oxotechnetium(V) typically include two bidentate chelating moieties or one tetradentate chelating moiety (having four chelating groups linked one to another) that altogether form, for example, diaminedithiols, monoamine-monoamidedithiols, triamide-monothiols, monoamine-diamide-monothiols, diaminedioximes, and hydrazines.

As discussed hereinabove, preferred metals according to the present embodiments include palladium, cobalt, nickel and copper. Palladium, cobalt and nickel are divalent metals and are hence typically present in an oxidized form thereof, namely, as Pd(II), Co(II) and Ni(II), respectively. These metals therefore form stable metal-coordination complexes with bidentate ligands that have the metal-coordination groups described hereinabove. The nature of metal-coordination groups utilized in the course of the process of depositing the metal coat of the protein's surface may affect the process efficiency. If the metal is poorly coordinated, an unstable complex is formed. The nature and structure of the metal-coordination groups may also exert a shielding which can affect the reduction by the reducing moiety.

The chelating moieties preferably have, in addition to the metal-coordinating group, at least one more functional group, referred to and discussed hereinbelow as the third functional group, which is utilized for its conjugation to the protein surface. As is discussed in detail hereinbelow, this functional group preferably forms a bond with reactive groups on the protein's surface.

Using the methodology devised for producing the metal-coated proteins presented herein, a stable protein-metal ion complex was successfully prepared, as demonstrated in the Examples section that follows.

Thus, according to another aspect of the present invention there is provided a composition-of-matter which comprises a protein having a modified surface and a plurality of ions of a metal attached to at least a portion of its surface and forming a protein-metal ion complex. The modified surface of the protein, according to this aspect, has a plurality of chelating moieties attached thereto, which are being for forming a complex with ions of the metal.

Although the number and location of the chelating groups can be finely controlled when utilizing the methodology presented herein, the chelating moieties, according to this aspect, are conjugated to the surface of the protein in large numbers, and cover a substantial part of its surface area. This plurality of chelating moieties allows for a corresponding plurality of metal ions to complex therewith and form the composition-of-matter presented in this aspect. This form of partial or total coverage of the surface of a protein with chelated metal ions, wherein the molar ratio of the protein to metal is in the order of one mol protein to at least several tens, and preferably several hundreds to several thousands mol metal atoms is substantially different than the attachments of one or few metal ions to one protein molecule in an attempt to tag the protein with a metal, wherein the molar ratio of the of the protein to metal is in the order of one to less than 10.

As is exemplified in the Examples section that follows, a modified protein having a plurality of chelating moieties attached to its surface was prepared by conjugating functional polymeric chains to the protein surface. Due to the utilization of such polymeric chain, the number of reactive groups generated on the protein surface reached a few hundreds and allowed to complex thereto a corresponding number of metal ions.

As is further exemplified in the Examples section that follows, by utilizing a modified protein having such a plurality (e.g., hundreds) of chelating moieties attached thereto, a continuous metal coat can be formed on the protein surface upon subsequent reduction of the metal ion-protein complex.

While the attachment of one or a few metal ions to the surface of antibodies is taught in European Patent No. EP00173629B1, this disclosure fails to teach an antibody having a plurality of metal ions, as in ten and hundreds thereof, attached thereto. Nevertheless and regardless of the dissimilar teachings in the art, the protein, according to preferred embodiment of this aspect of the present invention, is any protein as described hereinabove, excluding an antibody.

The metal-coated proteins described herein were successfully prepared and analyzed, and their preparation optimized, as presented in the Examples section that follows.

Thus, according to another aspect of the present invention, there is provided a process of preparing a metal-coated protein. The process, according to this aspect of the present invention, is effected by reacting the protein, having a characteristic biological attributes as discussed above, with one or more chelating moieties, to thereby obtain a modified protein having chelating moieties attached to at least a portion of its surface. As discussed in details hereinabove, these chelating moieties are being for forming a complex with ions of the metal.

The process is further effected by contacting this modified protein with a first aqueous solution containing ions of the metals presented hereinabove, to thereby obtain a solution containing a complex of the modified protein and the metal ions; and then contacting this complex solution with a first reducing agent, which is being for reducing the metal ions in-situ on the protein's surface, thus forming the metal-coated protein which substantially retains the original biological activity and/or dissolvability of the native, untreated protein.

Obtaining the modified protein having chelating moieties attached to its surface is preferably performed by firstly modifying the protein so as to have a plurality of reactive groups on its surface. These reactive groups are for conjugating the chelating groups thereto secondly.

As used herein, the phrase “reactive group” describes a chemical group that is capable of undergoing a chemical reaction that typically leads to a bond formation. The bond, according to the present embodiments, is preferably a covalent bond. Chemical reactions that lead to a bond formation include, for example, nucleophilic and electrophilic substitutions, nucleophilic and electrophilic addition reactions, addition-elimination reactions, cycloaddition reactions, rearrangement reactions and any other known organic reactions that involve a reactive group.

Hence, according to a preferred embodiment of this aspect of the present invention, the process is effected by generating such reactive groups to the protein surface, so as to form an activated protein in terms of the reactivity of its surface toward the conjugation described herein. Preferably, the reactive groups are selected capable of undergoing the conjugation reaction with the chelating moiety under mild conditions which will not abolish the protein functionally essential characteristics.

Representative examples of suitable reactive groups according to the present invention include, without limitation, amine, halide, carbonyl, acyl-halide, aldehyde, sulfonate, sulfoxide, phosphate, hydroxy, diol, alkenyl, alkoxy, aryloxy, thiohydroxy, thioalkoxy, thioaryloxy, cyano, nitrile, nitro, azo, isocyanate, sulfonamide, carboxylate, N-thiocarbamate, O-thiocarbamate, urea, thiourea, O-carbamate, N-carbamate, C-amide, N-amide, guanyl, guanidine and hydrazine, as these terms are defined herein.

The term “halide” and “halo” describes fluorine, chlorine, bromine or iodine.

The term “acyl halide” describes a —(C═O)R″″ group wherein R″″ is halide, as defined hereinabove.

As used herein, the term “aldehyde” refers to an —C(═O)—H group.

While some proteins exhibit some types of naturally occurring reactive groups that are capable of undergoing such chemical reactions under mild conditions, so as to conjugate chelating moieties thereto without affecting the protein essential characteristics, most of the proteins do not have such reactive groups.

According to a preferred embodiment of the present invention, a protein that therefore modified so as to have reactive groups on its surface while exploiting the presence of naturally occurring functional moieties that bear functional groups, as these phrases are defined hereinbelow, on the protein surface.

As used herein, the phrase “functional moiety” refers to a residue present on the surface of the subject protein, which preferably contains functional groups as defined hereinafter. Exemplary functional moieties, according to the present embodiments, include, without limitation amino acid residues, as well as post-translationally modified residues such as glycans, lipids, phospholipids, phosphates and the likes. Phosphate groups can be attached to a protein during a post-translational phosphorylation process by kinases. Reversible protein phosphorylation, principally on serine, threonine or tyrosine residues, is one of the most important and well-studied post-translational modifications.

As used herein, the phrase “functional group” describes a chemical group that has certain functionality and therefore can be subjected to chemical manipulations such as chemical reactions with other components which lead to a bond formation, oxidation, reduction and the like.

A variety of functional groups that can be utilized in the above-mentioned modification are available in proteins. These include, for example, functional groups derived from side chains of certain amino-acid residues, functional groups derived from the N-terminus or the C-terminus of the protein, and functional groups derived from residues that result from natural post-translational modification processes. Representative examples of such functional groups include, without limitation, amine, acyl, aldehyde, alkoxy, thioalkoxy, alkyl, alkenyl, C-amide, N-amide, carboxylate, diol, farnesyl, geranylgeranyl, guanidine, hydroxyl, thiohydroxy, imidazole, indole, phosphate and sulfate.

As used herein, the term “aldehyde” refers to an —C(═O)—H group. Naturally-occurring aldehydes on the surface pf proteins are rare and few, but can be in post-translationally modified proteins.

As used in the context of the present invention, the term “diol” refers to a vicinal diol which is a —CH(OH)—CH(OH)— group. Naturally-occurring diols on the surface pf proteins are frequently found in glycoproteins.

As used herein, the term “C-amide” refers to a —C(═O)—NR′R″ group, where R′ and R″ are as defined herein.

As used herein, the term “N-amide” refers to an —NR′C(═O)—R″ group, where R′ and R″ are as defined herein.

The term “farnesyl”, as used herein, refers to the fatty residue of fernesene, typically attached to post-translationally modified cysteine residues at the C-terminus of proteins in a thioether linkage (—C—S—C—).

The term “geranylgeranyl”, as used herein, refers to the fatty residue of geranylgeranene, typically attached to post-translationally modified cysteine residues at the C-terminus of proteins in a thioether linkage.

The term “guanidine” refers to a —NR′C(═NR″)—NR′″R* group, where R′ and R″ are as defined herein and R′″ and R* are defined as either R′ or R″. In the context of the present invention, guanidine is a functional group on the side-chain of the amino-acid arginine, therefore it is preferably —NH—C(═NH)—NH₂.

As used herein, the term “imidazole” refers to the five-membered heteroaryl group that includes two non-adjacent nitrogen atoms. An imidazole residue can be found in the side-chain of the amino acid histidine.

As used herein, the term “indole” refers to refers to a bi-cyclic heteroaryl comprised of fused phenyl and pyrrole groups. An indole residue can be found on the side-chain of the amino acid tryptophan.

As used herein, the term “sulfate” refers to a —O—S(═O)₂—O—R′, with R′ as defined herein. Modification of proteins with sulfate occurs typically at tyrosine residues, and the universal sulfate donor is 3′-phosphoadenosyl-5′-phosphosulphate.

Preferred functional groups according to embodiments of the present invention include, without limitation, amine, carboxylate, hydroxyl, thiol and aldehyde.

The conjugation reaction can be catalyzed by one or more enzymes so as to allow to perform a reaction, which generally requires harsh conditions, under mild conditions. Yet, for simplicity and effectiveness, the conjugation reaction is preferably performed in solution using no other proteins or other reagents which may complicate any stage of the process such as final purification. Thus, further preferably, the conjugation of the chelating moieties is effected via an existing or a modified functional group.

Such naturally occurring functional groups can be modified to other functional groups, which are more suitable for a conjugation reaction with a chelating moiety under conditions which preserve the protein's functions.

In cases where a suitable functional group, with respect to an available functional group on a desirable chelating moiety, is unavailable on the protein and a modification of a naturally occurring yet unsuitable functional group is unfavorable, or where the functional group is found in limited numbers on the protein, the modification of the protein is effected via a multifunctional compound.

Thus, in preferred embodiments, modifying a protein so as to have reactive groups on its surface is effected by reacting a plurality of naturally occurring functional groups on the surface of the protein with a compound having at least two functional groups, referred to herein as a first and a second functional group. The first functional group is selected capable of reacting with naturally occurring functional groups on the surface of the protein, and the second functional group constitutes the abovementioned reactive group.

Exemplary compounds having at least two functional groups (multifunctional compounds) include, without limitation, glutaraldehyde, polyglutaraldehyde and other polyaldehydes, malonic acid and other polycarboxyl acids, ethane-1,2-dithiol and other polythiols, 3-aminomethyl-pentane-1,5-diamine and other polyamines, malononitrile and other polynitriles, and polyfunctional compounds having mixed types of functional groups, such as, for example, 3-amino-propionic acid, 4-amino-butyryl chloride, diethyl iminodiacetate, triazine and the likes.

Regardless of the part and counterpart to be attached therebetween by a bond, namely the protein via a naturally-occurring or modified functional group, the reactive group via the first or second functional groups on the polyfunctional compound, or the chelating moiety via the third functional group, the bond forming reaction is preferably effected under mild conditions and between two chemically-corresponding functional groups. Thus, for non-limiting examples, a hydroxy group on one part and an amine on the counterpart or vice versa, are selected so as to form an amide; a carboxylate or acyl-halide and hydroxy are selected so as to form a carboxylate; two thiol groups are selected so as to form a disulfide, an isocyanate and a hydroxy are selected so as to form a carbamate; and a hydrazine and a carboxylic acid are selected so as to form a hydrazide, and so on.

Aldehydes are highly reactive groups even in physiological conditions, meaning they are highly suitable use as reactive groups according to preferred embodiments. Thus, preferably, the reactive group is an aldehyde.

Aldehydes can be readily generated on or introduced to a protein surface, under mild conditions that do not affect the protein nature, using various methodologies well-known and well-described in the art, which are presented briefly hereinbelow. According to a preferred embodiment of the present invention, the reactive group is aldehyde, and the process is effected by providing a protein that has a plurality of aldehyde groups on its surface.

Several processes known in the art can be used to modify a protein so as to have reactive aldehyde groups on its surface. One of the most common methods for introducing aldehydes to the surface of functional moieties is oxidation, by mild oxidizing agents, of vicinal diols present in glycan residues of glycan-containing proteins. Proteins having glycan residues on their surface (also known as glycoproteins) possess an abundance of diol groups, which readily undergo oxidation to aldehydes using mild oxidizing agents or enzymes. Provided that the protein of choice is a glycoprotein, it has a plurality of functional diol moieties that form a part of glycan residues on its surface. These diols can be readily modified to aldehyde groups by oxidizing vicinal diol groups present on the glycan surface residues. The oxidation reaction can be effected in the presence of mild oxidizing agents such as, but not limited to, periodic acid and salts thereof, paraperiodic acid and salts thereof, and metaperiodic acid and salts thereof.

This methodology can further be utilized for generating aldehyde groups on the surface of a lipoprotein. Thus, functional alkenyl residues that form a part of functional moieties such as unsaturated fatty acids, ceramides or other lipids that may be present on a lipoprotein surface can be converted to glycols by osmium tetroxide and subsequently oxidized by any of the oxidizing agents cited above to aldehydes.

Furthermore, functional groups such as hydroxyl groups, that from a part of functional moieties such as N-terminal serine and threonine residues of peptides and proteins can be selectively oxidized by periodate to aldehyde groups.

Alternatively, aldehydes can be introduced to specific cites on a protein surface be means of galactose oxidase. Galactose oxidase is an enzyme that oxidizes terminal galactose residues that are typically present in glycoproteins, to aldehydes. Another common method of introducing aldehydes to the protein surface is by conjugation of a polyaldehyde to chemically compatible functional groups on the protein surface.

As mentioned above, aldehydes are highly suitable reactive groups, thus preferably, the first functional group can be any of the above-mentioned functional groups, and the second functional group is an aldehyde.

As is well known and described in the art, conjugation of aldehydes to amine groups that form a part of a protein results in the formation of Schiff bases (imines). This reaction can be carried under mild conditions that do not affect the protein essential characteristics (see, for example, U.S. Pat. No. 4,904,592).

Since amines represent an exemplary preferred reactive functional group which naturally occur on the surface of proteins, and since aldehydes readily react with amines, the preferred first functional group is also an aldehyde. These preferred embodiments constitute a polyaldehyde compound, having at least two aldehyde groups, one for forming a bond with the protein and one for forming a bond with the chelating moiety.

As used herein, the term “polyaldehyde” describes a compound that has at least two free aldehyde groups.

Representative examples of polyaldehydes that are suitable for use in this context of the present invention include glutaraldehyde and its polymeric derivatives, which are referred to herein as polyglutaraldehyde. When a polyaldehyde such as polyglutaraldehyde is used in such a reaction, one of the free aldehyde groups is reacted so as to form the Schiff base, while at least one other aldehyde group, constituting the reactive group, remains free yet attached to the amine.

According to preferred embodiments, the functional group on the protein surface is an amine group, which forms a part of lysine residues which typically protrude from the surface of the protein, and can be readily modified using mild conditions. Another amine group which can be employed for that purpose is the amine at the N-terminus of the protein.

Apart from aldehydes, other exemplary groups which react readily with amines include, without limitation, carboxyl, acyl, alkene and the likes.

Thus, according to another preferred embodiment of the present invention, a protein having a plurality of aldehyde groups on its surface is obtained by reacting functional groups such as amine groups, which form a part of functional moieties such as lysine residues and/or the N-terminus of the protein with a polyaldehyde. Such a reaction leads to the formation of free aldehyde groups that are attached to the protein surface via imine bonds.

It should be noted that a modified protein which has more than one type of a reactive group can be prepared and utilized in this and other aspects of the present invention. Such a modified protein is prepared by stepwise modifications of naturally occurring functional moieties that are present on its surface, using, for example, the methodologies described hereinabove and other well established processes known in the art.

It should further be noted that reactive groups can be placed at one or more specific sites on the surface of the protein, so as to direct the metal deposition to preferred locations. This site-directed metal deposition can determine the physical as well as biochemical properties of the resulting composition-of-matter presented herein, such as, for example, its biological activity and electrical conductivity.

As demonstrated in the Examples section that follows (see, Example 1), the present inventors used the available lysine-stemming amines on an exemplary protein, the enzyme glucose oxidase, and polyglutaraldehyde to modify the protein. This protein is known to have about 30 lysine residues which naturally occur in the polypeptide chain thereof. The polyglutaraldehyde compound used for the modification of the enzyme exhibited an average of more than 10 aldehyde groups. Therefore, a rough estimation of the total number of aldehyde reactive groups present on the surface of the exemplary protein upon its modification is 300.

Hence, according to preferred embodiments of the present invention, the number of reactive groups which can be added to a protein ranges from about 5 reactive groups to about 1000 reactive groups, preferably from about 100 to about 1000 reactive groups. By selecting a suitable functionalized polymeric substances, higher numbers of a few thousands of reactive groups can also be generated.

The ability to finely control the amount of reactive groups, available for conjugation with a chelating moiety, consequently allows to finely control the amount of metal which would be deposited onto the surface of the protein. This control is crucial for enabling the maintenance of the protein's specific biological characteristics and dissolvability, and also the metallic characteristics of the resulting metal-coated protein. An uncontrollable metal deposition could result in an insoluble metallized protein, and inactive protein due to deformation, active-site blockage, denaturation or otherwise loss of characteristic features thereof. On the other hand, uncontrollable metal deposition could result in an insufficient metal deposition, rendering the resulting metal-coated protein useless in certain applications.

Once the protein has been modified, the chelating moieties can be conjugated to the reactive groups. As mentioned above, the chelating moieties have at least one metal-coordinating group, as discussed in detail hereinabove, and a third functional group, which is used to conjugate with the reactive group, namely forming a bond between the protein and the chelating moiety.

The third functional group is selected so as to be capable of forming a bond with a reactive group under mild conditions so as not to affect the biological activity of the protein. Exemplary functional groups serving as the third functional group include, without limitation, amine, carbonyl, aldehyde, alkoxy, thioalkoxy, alkyl, alkenyl, C-amide, N-amide, carboxyl, hydroxyl, thiohydroxy, phosphate sulfate, halide, cyano, isocyanate, nitro, acyl halide, azo, peroxo hydrazine, hydrazide, hydroxylamine, isocyanate, phenylhydrazine, semicarbazide and thiosemicarbazide.

The term “isocyanate” describes an —N═C═O group.

The term “nitro” describes an —NO₂ group.

The term “azo” or “diazo” describes an —N═NR′ with R′ as defined hereinabove.

The term “peroxo” describes an —O—OR′ with R′ as defined hereinabove.

As used herein, the term “hydrazine” describes a —NR′—NR″R′″ group, wherein R′, R″ and R′″ are each independently hydrogen, alkyl, cycloalkyl or aryl, as these terms are defined herein.

The term “hydrazide”, as used herein, refers to a —C(═O)—NR′—NR″R′″ group wherein R′, R″ and R′″ are each independently hydrogen, alkyl, cycloalkyl or aryl, as these terms are defined herein.

As used herein, the term “hydroxylamine” refers to a —NR′—OH group, where R′ is as define herein.

As used herein, the term “phenylhydrazine” refers to an —NR′—NR″R′″ group, where R′, R″ and R′″ are as define herein, with at least one of R′, R″ and R′″ being an aryl, as this term is defined herein.

As used herein, the term “semicarbazide” refers to a —NR′—(═O)NR″—N R″#R* group, and the term “thiosemicarbazide” refers to a —NR′—C(═S)NR″—NR″R* group, where R′, R″, R′″ and R* are define herein.

In cases where the reactive group is an aldehyde, the third functional group is preferably an amine which can readily form a Schiff-base with an aldehyde reactive group, as discussed hereinabove.

Other functional groups which can serve as a third functional group, according to the present invention, by reacting with an aldehyde group include, without limitation, carboxyl, acyl, hydrazine, hydrazide, hydroxylamine, isocyanate, phenylhydrazine, semicarbazide and thiosemicarbazide.

Hence, a modified protein having at least one chelating moiety conjugated thereto, selected suitable for forming a complex with the desired metal ions, is provided, preferably by the process described hereinabove.

The process, according to this aspect of the present invention, is further effected by contacting the modified protein with a solution containing the metal ions, referred to herein as the first aqueous solution. The ions interact with the chelating moieties to form a complex of the modified protein and the metal ions, and thereby become attached to the surface of the protein.

Together with the previously discussed number of reactive groups, which determines the number of sites on the protein capable of forming a metal-ion complex, another key factor in controlling the amount of metal ions which would be attached to the modified protein is the concentration of the first metal ion aqueous solution. This controllability is illustrated in FIG. 2, and demonstrated in the Examples section that follows hereinbelow (see, Example 2). According to preferred embodiments, the concentration of the metal ions in the first aqueous solution ranges from about 0.1 mM to about 10 mM. Preferably the concentration of the metal ions in the first aqueous solution ranges from about 0.2 mM to about 5 mM, and most preferably the concentration of the metal ions in 2 mM.

In order to prevent, or otherwise minimize the reduction of unbound metal ions in the solution by the reducing agent, the process according to this aspect may further be effected by filtering the solution containing the complex prior to contacting the complex with the reducing agent.

As discussed hereinabove, the in-situ reduction of the metal ions in the complex is effected by contacting the solution containing the complex with a reducing agent, referred to herein as the first reducing agent, to thereby obtain the metal-coated protein according to the present invention.

The reducing agent, according to the present embodiments, reduces metal ions that are complexed to the modified protein described herein to elemental metal atoms.

As used herein, the phrase “reducing agent” refers to a chemical substance that is capable of participating in a reduction/oxidation process by either directly or indirectly inducing reduction of other components that participate in such a process. Preferred reducing agents, according to the present embodiments, are selected capable of inducing reduction of ions of the desired metal into elemental metal atoms. More preferred reducing agents are chemical substances that can affect such a reduction under mild conditions (e.g., physiological conditions) and therefore do not adversely affect functionally essential characteristics of the protein.

Some of the most commonly used reducing agents for electroless deposition of, for example, palladium, nickel, copper and cobalt, include hypophosphite (H₂PO₂⁻) and dimethylamineborane ((CH₃)₂HN.BH₃). Other commonly used reducing agents include, without limitation, dimethylamineborane, azide, borane-dimethyl sulfide, borane-tetrahydrofuran, decaborane, diborane, formaldehyde, formate, hydrazine, hydrazoic acid, hyposulfite, phosphites, sulfite, sulfoxylate, tartrate and thiosulfate.

Hypophosphite, a preferred reducing agent according to the present embodiments, is considered a highly stable and very potent reducing agent in almost any pH range as long as there are no oxidants in the reaction media. It can even reduce metal salts such as gold, silver and platinum salts and deposit them as metallic elements while turning into a phosphite (HPO₃²⁻). It is further characterized as non-toxic, non-hazardous and environmental-friendly.

One proposed model for the reduction mechanism of divalent metal ions using these reducing agents, such as hypophosphite, involves the catalytic dehydrogenation of the reducing agent which is coupled to a hydride transfer and reaction thereof with the metal ions to form elemental metal atoms.

Scheme 1 below presents the proposed mechanism for metal reduction using hypophosphite.

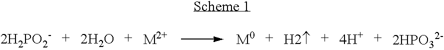

As discussed hereinabove, the metal-coated protein may have more than one type of metals comprising the coat. The second (and third, fourth etc.) metal, can be added either before or after reducing the first metal in complex with the protein.

One proposed mechanism for the catalytic effect of specific divalent metal ions, namely the first metal such as nickel and palladium, on the reduction of other metals, namely the second (third, fourth etc.) metal is presented in Scheme 2 below. The mechanism proposes that the first metal forms nucleation sites of the reduced catalytic metal onto which other metals, such as copper, can be reduced and deposited.

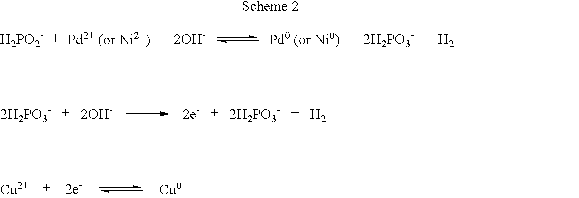

Since the reduction process is effected on metal ions which are bound to the protein via the chelating groups, the initiation and propagation of the reductive oxidation process of the metal ions in solution will take place substantially at the surface of the protein wherein the catalytic divalent metal ions are held in place, or in the immediate vicinity thereof.

Therefore, according to preferred embodiments, the process is further effected by either contacting the solution containing the complex with a second aqueous solution containing ions of a second metal concomitantly with the first reducing agent, or contacting the metal-coated protein with a second aqueous solution containing ions of a second metal in the presence of a second reducing agent subsequent to adding the first reducing agent. The second reducing agent is for reducing the second metal ions. This process affords a metal-coated protein having an additional metal coating which comprises the second metal, as demonstrated in the Examples section that follows (see, Example 3).

The first and second reducing agents may be the same substance, added in two separate steps, or two different substances. Similarly, the first and second aqueous solutions can contain ions of the same metal or of different metals.

The second metal can be added in a second aqueous solution, preferably having a concentration which ranges from about 0.1 mM to about 10 mM. Preferably the concentration of the metal ions in the second aqueous solution ranges from about 0.2 mM to about 5 mM, and most preferably the concentration of metal ions in 2 mM. This concentration affects the molar ratio of the protein to metal, as discussed hereinabove and can further be seen in the Examples section that follows (see, Table 2, Example 3).

Being biologically active and dissolvable in aqueous solutions, the composition-of-matter according to the present invention, comprising the metal-coated proteins, can be utilized in pharmaceutical applications. These include, for example, antimicrobial preparations, particularly when the metal has biocidal activity.

Thus, according to another aspect of the present invention, there is provided a pharmaceutical composition comprising, as an active ingredient, the composition-of-matter presented herein and a pharmaceutically acceptable carrier.

In a preferred embodiment, the pharmaceutical composition is an antimicrobial preparation, useful in the treatment of a bacterial and/or fungal infection, Such pharmaceutical compositions preferably comprise a composition-of-matter of a protein coated by a biocidal metal.

Biocidal metals which can be beneficially used in the context of this aspect include, without limitation, silver, copper, zinc, mercury, tin, lead, bismuth, cadmium, chromium, cobalt, nickel and any combination thereof.

In one embodiment of this aspect of the present invention, the pharmaceutical composition comprises a composition-of-matter that includes a metal-coated hydrogen peroxide producing enzyme, such as, for example, glucose oxidase, and is identified for use in the treatment of bacterial and fungal infections.

As used herein a “pharmaceutical composition” refers to a preparation of the metal-coated enzyme described herein, with other chemical components such as pharmaceutically acceptable and suitable carriers and excipients. The purpose of a pharmaceutical composition is to facilitate administration of a compound to an organism.

Hereinafter, the term “pharmaceutically acceptable carrier” refers to a carrier or a diluent that does not cause significant irritation to an organism and does not abrogate the biological activity and properties of the administered compound. Examples, without limitations, of carriers are: propylene glycol, saline, emulsions and mixtures of organic solvents with water, as well as solid (e.g., powdered) and gaseous carriers.

Herein the term “excipient” refers to an inert substance added to a pharmaceutical composition to further facilitate administration of a compound. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences” Mack Publishing Co., Easton, Pa., latest edition, which is incorporated herein by reference.

Pharmaceutical compositions for use in accordance with the present invention thus may be formulated in conventional manner using one or more pharmaceutically acceptable carriers comprising excipients and auxiliaries, which facilitate processing of the metal-coated enzymes into preparations which, can be used pharmaceutically. Proper formulation is dependent upon the route of administration chosen. Toxicity and therapeutic efficacy of the metal-coated proteins described herein can be determined by standard pharmaceutical procedures in experimental animals, e.g., by determining the EC₅₀, the IC₅₀ and the LD₅₀ (lethal dose causing death in 50% of the tested animals) for a given metal-coated protein. The data obtained from these activity assays and animal studies can be used in formulating a range of dosage for use in human.

The dosage may vary depending upon the dosage form employed and the route of administration utilized. The exact formulation, route of administration and dosage can be chosen by the individual physician in view of the patient's condition. (See e.g., Fingl et al., 1975, in “The Pharmacological Basis of Therapeutics”, Ch. 1 p. 1).

The amount of a composition to be administered will, of course, be dependent on the subject being treated, the severity of the affliction, the manner of administration, the judgment of the prescribing physician, etc.

Compositions of the present invention may, if desired, be presented in a pack or dispenser device, such as an FDA (the U.S. Food and Drug Administration) approved kit, which may contain one or more unit dosage forms containing the active ingredient. The pack may, for example, comprise metal or plastic foil, such as, but not limited to a blister pack or a pressurized container (for inhalation). The pack or dispenser device may be accompanied by instructions for administration. The pack or dispenser may also be accompanied by a notice associated with the container in a form prescribed by a governmental agency regulating the manufacture, use or sale of pharmaceuticals, which notice is reflective of approval by the agency of the form of the compositions for human or veterinary administration. Such notice, for example, may be of labeling approved by the U.S. Food and Drug Administration for prescription drugs or of an approved product insert. Compositions comprising a metal-coated protein of the invention formulated in a compatible pharmaceutical carrier may also be prepared, placed in an appropriate container, and labeled for treatment of an indicated condition or diagnosis, as is detailed hereinabove.

Thus, according to an embodiment of the present invention, depending on the selected components of the metal-coated enzymes, the pharmaceutical compositions of the present invention are packaged in a packaging material and identified in print, in or on the packaging material, for use in the treatment of bacterial and/or fungal infections, as described hereinabove.

The preparation of biologically active metal-coated hydrogen peroxide producing enzymes using the methodologies described herein, particularly when comprising a biocidal metal, may therefore be beneficially utilized in the treatment of bacterial and/or fungal infections. As is delineated hereinabove, such metal-coated enzymes are capable of exerting a synergistic effect as a result of the generation of hydrogen peroxide, an anti-microbial agent by itself, which may further act as an oxidizing agent that may oxidize in its immediate vicinity the metal deposited on the enzyme and thus generate free metal ions. The released biocidal metal ions and the generated hydrogen peroxide may thus act synergistically as toxic agents against various bacteria, fungi and other microorganisms.

Hence, according to another aspect of the present invention, there is provided a method of treating bacterial and/or fungal infections. The method, according to this aspect of the present invention, is effected by administering to a subject in need thereof a therapeutically effective amount of a composition-of-matter, preferably including a metal-coated hydrogen producing enzyme, as described hereinabove.

As used herein and is well known in the art, hydrogen peroxide producing enzymes are enzymes which catalyze reactions during which hydrogen peroxide is generated. Representative examples of such enzymes include, without limitation, glucose oxidase, oxalate oxidase and superoxide dismutase.

As used herein, the terms “treating” and “treatment” includes abrogating, substantially inhibiting, slowing or reversing the progression of a condition, substantially ameliorating clinical or aesthetical symptoms of a condition or substantially preventing the appearance of clinical or aesthetical symptoms of a condition.

As used herein, the phrase “therapeutically effective amount” describes an amount of the composite being administered which will relieve to some extent one or more of the symptoms of the condition being treated.

According to a preferred embodiment of this aspect of the present invention, the substrate of the hydrogen peroxide producing enzyme is a vital food source, such as sugars, or other metabolites crucial for the survival of the target bacteria or fungi. Using such an enzyme provides an additive effect since depleting a vital source that is required for the bacteria or fungi growth further results in growth inhibition thereof. Hence, altogether, using such a metal-coated enzyme results in a triple action against infectious microorganisms: a toxic effect exerted by the hydrogen peroxide produced during the enzymatic catalysis of the enzyme, a toxic effect exerted by biocidal metal ions that are released when the metal-coated enzyme interacts with the produced hydrogen peroxide, and a growth inhibition of the microorganisms that results from depleting a vital source thereof.

Thus, preferred metal-coated enzymes according to this aspect of the present invention are biocidal metal-coated hydrogen peroxide producing enzymes that act on a substrate that serves as a vital source for microorganism growth. An example for such a substrate is sugar, e.g., glucose. A preferred enzyme for use in this context is therefore a hydrogen-producing enzyme that uses glucose as a substrate. An exemplary and preferred enzyme, according to this aspect of the present invention, is glucose oxidase.

The metallic nature of the deposited metal, namely chemical and physical attributes which are characteristic to metals, such as electronic and heat conductivity and magnetism, on the metal-coated proteins described herein, along with the biological specificity typically associates with biological active proteins, can be further harnessed in the construction of various conductors and semiconductors elements. The ability to combine the nano-size metal particles deposited on a biologically active protein, and the natural molecular recognitions and highly-specific chemical binding capacities of proteins, presents an opportunity to develop nano- and micro-sized electronic circuit assemblies which are assembled by using, partially or entirely, the natural affinity of proteins to other proteins and ligands. As used herein, the term “nano-size” refers to a size magnitude that ranges from 1 nm to 1000 nm.

Hence, according to yet another aspect of the present invention, there is provided a metallic element which includes the composition-of-matter described above, namely a metal-coated protein. The metallic element, according to this aspect of the present invention, preferably has a size magnitude which ranges between 1 nanometer and 1000 nanometers.

The metal, according to this aspect, is preferably a conductive metal or a semi-conductive metal, and/or a magnetic or magnetizable metal.

The term “conductive” or “conductor” as used herein refers to materials, and in the context of the invention preferably metals, that contain delocalized and thus transferable electrons, transferable ions, or otherwise transferable electrical charges. In the context of metals, an electric potential difference applied across separate points on a conductor, the electrons of the metal are forced to move, and an electric current between those points can be detected.

The term “magnetic” as used herein refers to a physical characteristic of a substance which exhibits itself by producing a permanent magnetic field, thereby showing an aptitude to attract ferromagnetic substances, such as iron, and align in an external magnetic field. Proteins coated with a magnetic metal in the context of the present invention, are nano-sized magnets, and can be utilized as such in applications which utilize the combination of biological activity and magnetic characteristic.

The term “magnetizable” refers to a physical characteristic of a substance which can be turned into a permanent or a temporary magnetic substance by induction or by electrical field which is applied thereon.

The metallic element, according to preferred embodiments, can take the shape of a naturally-occurring self-assembled structure comprising naturally-occurring proteins. Hence, according to preferred embodiments, the protein comprising the metallic element forms a part of a self-assembled structure, which is composed of a plurality of this protein.

Since the metal-coated proteins present herein preserve their native structure and activity substantially, the metallization process can be effected before the structure self-assembles. Alternatively the metallization can be effected after the self-assembly process.

An exemplary metallic element is a coil, as in an electric circuit. A coil has one or more turns, roughly circular or cylindrical, and typically made of conductive metal wire. It is designed to produce a magnetic field or to provide electrical resistance or inductance (choke coil). If a soft iron core is placed within the coil, passage of an electric current in the coil will produces an electromagnet. In order to form a nano-sized electric coil, as described above, one can make use of a naturally-occurring biological proteinous structure. An exemplary self-assembled proteinous structure suitable as a coil template is the capsid (proteinous viral coat) of the tobacco mosaic virus (TMV), and the corresponding protein, according to this embodiment, is its capsomere. A capsomere is a protein-based subunit of a viral capsid, designed to have strong affinity to other identical capsomeres so as to form a particular structure and, upon reaching a minimal number of subunits, self-assemble to form that structure, namely the capsid. The capsid of the TMV has a cylindrical rod shape of about 300 nm in length and 15 nm in diameter, sheathing the viral RNA therein. The capsomere are arranged in a tight spiral structure, coiling with the RNA strand they are attached to. According to this embodiment, the capsomeres can be specifically modified so as to have a metal-coat in surface areas which do not hinder the capsid formation. These metal-coated capsomeres can be allowed to self-assemble (in the presence of the viral RNA), thereby forming a nanosized metallic coil, having the shape and dimensions of the TMV capsid. Alternatively, the caspid can be metallized after it has assembled, again resulting in a nanosized electrical coil.

These conductive element based on metal-coated proteins can be used, according to another aspect of the present invention, in the construction of electronic circuit assemblies comprising an arrangement of conductive elements interconnecting many other electronic elements wherein some are the composition-of-matter described above.

Devices that require nanosized electronic circuitry and other nanosized metallic, conductive and/or magnetic elements can be constructed, according to yet another aspect of the present invention, using the metal-coated proteins presented herein.

Such devices can comprise, for example, a nanosized or a macrosized switch which is designed to employ a naturally occurring biological affinity pair to effect the generation of a signal, such as an electrical or magnetic signal upon binding of the members of the affinity pair. Exemplary affinity pairs include antibody-antigen affinity pairs, receptor-ligand affinity pairs or any other affinity pair such as the avidin-biotin affinity pair. The signal is generated by immobilizing one member of the affinity pair near or on a signal detector, and allowing the conductive and/or magnetic metal coated-second member to bind thereto, thereby the signal is generated and detected.

A signal detecting device, such as described hereinabove, which can beneficially employ the unique characteristics of metal-coated proteins, and especially metal-coated enzymes is, for example, an electrode, and as derived from that, the composition-of-matter described herein can be further utilized in the construction of biosensors based on electrodes having a metal-coated protein, such as an enzyme attached thereto, for the determination of an analyte in a sample.

For example, micro- and nano-electrodes for the quantitative and qualitative detection of glucose is an important technological goal on the path to produce small and low-cost glucose meters which are in high demand as medical and research devices. The presently known systems that utilize glucose oxidase in bio-electrodes aimed at detecting glucose concentrations in a sample are typically prone to high noise level and interferences from other electro-oxidizable species. Other systems involve the cost-ineffective use of bi-enzymatic systems.

While further reducing the present invention to practice, an electrochemical biosensor system capable of quantitatively and qualitatively detecting glucose was constructed and successfully practiced, as demonstrated in the Examples section that follows (see, Example 5). This glucose detecting biosensor is based on an electrode having a palladium or cobalt-coated glucose oxidase deposited thereon and is further based on the amperometric electrochemical measurement of the current resulting from the electrochemical oxidation or reduction of an electroactive species at a constant applied potential.

Thus, according to another aspect of the present invention there is provided an electrode which comprises, as a signal generating component, a composition-of-matter as described herein being deposited thereon.

The electrode having the composition-of-matter deposited thereon is referred to herein as the working electrode, as this term is commonly used in the art. The basis of the working electrode, according to the present invention, can be any commercially available or specially prepared working electrode. The most commonly available working electrodes are carbon-based, such as, for example working electrode made of glassy carbon, pyrolytic carbon and porous graphite. Working electrode based on metals, such as, for example, platinum, gold, silver, nickel, mercury, gold-amalgam and a variety of alloys, can also be used as working electrode according to the present invention. Preferably the working electrode is a carbon-based working electrode.

The composition-of-matter can be deposited onto the working electrode by means of, for example, a sol-gel film, a polymer film, a cross-linking agent and/or other protein immobilization techniques known in the art. Preferably the immobilization of the composition-of-matter is effected by a cross-linking process using glutaraldehyde as a cross-linking agent. The cross-linked structure prevents the composition-of-matter presented herein from eluting into a liquid sample.

Biosensors are based on technology that can respond to physical stimuli and the capacity to amplify, display and record this response in a qualitative and/or quantitative and human-readable format, thus effecting the detection of an analyte in a test-sample that combines a biological component with a physicochemical detector component.

Typically biosensors comprise a sensitive biological element such as, for example, an enzyme, an antibody, a nucleic acid, a cell receptor, an organelle, a microorganism, a tissue and the likes, or derivatives thereof; a transducer element, which converts input energy into output energy and an be also a biological component or a derivative thereof; and a physicochemical detector element which can effect the detection task, for example, optically, electrochemically, magnetically, thermometrically or piezoelectrically.

Various biosensors can gain effectiveness from the composition-of-matter presented herein by employing a metal-coated protein. For example, an optically-based biosensing technology, known as surface plasmon resonance (SPR), utilizes a layer of gold having a first member of a biological affinity-pair attached to its surface. A measurable signal is detected as a change in the absorption of laser light caused by electron waves (surface plasmons) in the gold upon binding of the second member of the affinity-pair, the target analyte, to first member on the gold surface. An SPR biosensor having the surface-attached member of the affinity-pair coated with a metal would effect a stronger signal and thus constitute a more sensitive SPR biosensor.

Similarly, other biosensors which are based on the binding of one biologic member of an affinity-pair to an immobilized counterpart thereof could gain efficiency in signal detecting if one member is metallized. For example, magnetically based biosensors can be developed on the basis of generating a magnetic signal with a magnetic metal coat over one or more portentous component thereof.

The most wide-spread and developed biosensors are electrochemically based biosensors. These are typically based on enzymatic reaction that produces electron transfers. Biosensors typically comprise a reference electrode, an active working electrode and a sink (counter) electrode. The analyte is involved in the reaction that takes place on the working electrode surface, and the electrons/ions produced create a detectible current signal.

The electrode described herein can be utilized for constructing a biosensor system for electrochemically detecting analytes in a liquid sample.

As used herein throughout, the term “detecting” encompasses qualitatively and/or quantitatively determining the level (e.g., concentration, concentration variations) of an analyte in the sample.

Hence, according to another aspect of the present invention there is provided a biosensor system for electrochemically determining a level of an analyte in a liquid sample, which comprises an insulating base and an electrode system. The electrode system, according to the present invention, includes the abovementioned working electrode, whereby the composition-of-matter described herein comprises a metal-coated protein which is capable of reacting with the analyte (e.g., a substrate) while producing a transfer of electrons.

The biosensor presented herein is based on typical biosensors known and used in the art, and includes an electrodes system in an insulating base. The electrodes system, preferably made of carbon electrodes, includes a working electrode having the composition-of-matter presented herein deposited thereon, and a counter (also referred to as an auxiliary electrode) electrode. The electrode system can further include a reference electrode, such as, for example, a saturated calomel electrode.

As in typical biosensors, when the biosensor is placed in contact with a liquid sample containing the analyte, the analyte electrochemically reacts with metal-coated protein deposited on the working electrode, so as to produce a transfer of electrons (en electric current). The presence and magnitude of the electric current, which is proportional to the concentration of the analyte in the liquid sample, is recorded by the system.

The biosensor of the present invention can include any of the compositions-of-matter described herein, as long as the protein in the composition-of-matter can react with an analyte and the reaction can be electrochemically detected. Preferred compositions-of-matter, however, are those containing an enzyme as the metal-coated protein and more preferably an oxidoreductase (redox) enzyme.

The term “analyte” as used herein refers to a substance that is being analyzed for its level, namely, presence and/or concentration, in a sample. An analyte is typically a chemical entity of interest which is detectable upon an electrochemical reaction and which the biosensor presented herein is design to detect. Examples of analytes that are typically detectable by biosensors include, without limitation, enzyme substrates. A level of an enzyme substrate analyte in a sample is determined by biosensors that include metal-coated enzymes, whereby this level is a function of the electric current produced upon the enzymatic reaction.

The term “redox” as used herein refers to a chemical reaction in which an atom in a molecule or ion loses one or more electrons to another atom or ion of another molecule.

The phrase “oxidoreductase enzyme”, which is also referred to herein interchangeably as “redox enzyme” describes an enzyme which catalyzes a reaction that involves the transfer of electrons from one molecule (the oxidant, also called the hydrogen donor or electron donor) to another molecule (the reductant, also called the hydrogen acceptor or electron acceptor), or, in short, catalyzes a redox reaction. Examples of redox enzymes include, without limitation, glucose oxidase, glucose dehydrogenase, lactate oxidase, lactate dehydrogenase, fructose dehydrogenase, galactose oxidase, cholesterol oxidase, cholesterol dehydrogenase, alcohol oxidase, alcohol dehydrogenase, bilirubinate oxidase, glucose-6-phosphate dehydrogenase, amino-acid dehydrogenase, formate dehydrogenase, glycerol dehydrogenase, acyl-CoA oxidase, choline oxidase, 4-hydroxybenzoic acid hydroxylase, maleate dehydrogenase, sarcosine oxidase, uricase, and the like.

When using a biosensor based on a hydrogen peroxide-producing enzyme to measure an analyte which is a substrate thereof, the oxidation current of H₂O₂ is usually proportional to the concentration of the analyte in solution and is detected at +700 mV versus a reference electrode. However, as mentioned above, monitoring hydrogen peroxide is limited by the presence of substances such as ascorbic acid and uric acid, which are electroactive at similar voltages and are abundant in physiological samples, such as blood serum, and would therefore interfere with amperometric transducers based on the O₂/H₂O₂ electron-transfer mediator system.

In order to overcome these limitations, non-physiological electron transfer mediators such as, for example, phenazines, tetrathiafulvalene (TTF), ferrocenes, ferrocyanides, quinones, fullerenes and ruthenium complexes are used, as is detailed hereinabove. Thus, the biosensor system presented herein preferably further comprises an electron transfer mediator (also referred to herein as a mediator). Preferably the mediator is a ferrocene derivative, and more preferably the mediator is ferrocene monocarboxylic acid.

Generally, all proteins, preferably enzymes and more preferably redox enzymes, can undergo the treatment of metallization as presented herein and exemplified in the Examples section that follows, and be coated with a single or multiple coats of a metal, such as silver, so as to form a coat of crystalline or amorphous silver thereon.

Preferably, the composition-of-matter deposited on the electrode used in the biosensor presented herein includes glucose oxidase, and hence the biosensor is preferably used for determining the level of glucose in a liquid sample.

Use of the metal-coated enzyme presented herein, such as, for example, palladium-coated glucose oxidase which includes an active enzyme having lysine-bound polyglutaraldehyde coupled to chelating moieties, offers several added advantages to an electrochemical system. These include, for example, stabilization of the metal-coated enzyme by its cross-linking with polyglutaraldehyde, hence prolonging the time of effective use of the electrode, and providing additional “wiring” between the metal-coated enzyme and the electrode. In addition, the crystalline morphology of the palladium coating of the enzyme provides a continuous contact surface between the enzyme and the working electrode, providing shorter distance for the ferrocene mediator to shuttle its electrons. Hence, another key advantage gained by using the metal-coated enzymes of the present invention for electrochemical electrodes is a significant increase in the total surface area of the electrode, as each metal-coated glucose oxidase molecule may be considered as an individual nano-electrode.

Therefore, according to preferred embodiments, the protein in the composition-of-matter is the enzyme glucose oxidase.

The biosensor presented herein is therefore designed for detecting an analyte in a sample, which can be, for example, a physiological sample extracted from an organism. Hence, according to another aspect of the present invention there is provided a method of electrochemically determining a level of an analyte in a liquid sample. The method, according to this aspect of the present invention, is effected by contacting the biosensor system presented herein with the liquid sample and measuring the transfer of electrons formed upon the electrochemical reaction between the analyte and the metal-coated protein, thereby determining the level of the analyte substrate in the sample. Use of a reference and/or use of a set of known standard samples with known concentrations can be used to convert the amperometric results into concentration of the analyte in the sample.

Preferably, the method presented herein is used for determining the level of glucose in a liquid sample, while utilizing metal-coated glucose oxidase.

However, by selecting a protein that can electrochemically react with an analyte so as to produce a transfer of electrons, and depositing such a metal-coated protein on a working electrode in a biosensor system, the systems and methods described herein can be further utilized for determining levels of versatile analytes.

Thus, several other important biochemical analytes can also be readily detected using the biosensors presented herein. Non-limiting examples include a biosensor for lactate using metal-coated lactate dehydrogenase, a biosensor for bilirubin using metal-coated bilirubin oxidase, and a biosensor for amino acids and peptides using metal-coated amino acid oxidase and tyrosinase. Other examples of enzymes which can be utilized by present invention are provided in Table 1 below, presenting the name of the enzyme which also indicates the analyte, i.e., substrate thereof, the chemical species that is formed in the course of the enzymatic reaction, and a typical exemplary use of the biosensor which can be constructed using these enzymes.

TABLE 1

| | Molecule generated or | |
|---|---|---|
| Enzyme/Ligand | captured | Use |
| Peroxidase | Hydrogen peroxide | Immunology, medicine |
| | | Environment |
| Glucose oxidase | Glucose | Medicine, Food industry |
| Alcohol oxidase | Alcohol | Food, medicine, police |
| Cholestrol oxidase | Cholesterol | Medicine, food |
| Choline oxidase | Choline, acetyl choline | Medicine, environment, |
| | | anti-warfare detector |
| Phenol oxidase | Phenol | Medicine, food, environment |
| Aminoacid oxidase | Amino acids | Medicine |
| Alcohol dehydrogenase | Alcohol, NAD | Food, medicine, police |
| Glucose dehydrogenase | Glucose, NAD | Medicine, Food industry |
| α and β-Glactosidase | Lactose, p-aminophenol -D | Food, molecular biology, cell |
| | galactopyranoside | markers, medicine, detection of |
| | | bacteria |
| α and β Glucosidase | Glucose, p-aminophenol -D | Food, molecular biology, cell |
| | glucopyranoside | markers, medicine, detection of |
| | | bacteria |
| α and β Glucoronidase | Glucoronic acid, | Food, molecular biology, cell |
| | p-amino-phenol -D | markers, medicine, detection of |
| | glucoronopyranoside | bacteria |
| Alkaline phosphatase | Organic phosphate | Immunology, Food, molecular |
| | | biology, cell markers, medicine, |
| | | detection of bacteria |

The biosensors presented herein can be further utilized for monitoring of drugs. Such biosensors include, for example, a biosensor for theophylline using metal-coated theophylline oxidase. In addition to medical applications, biosensors based on the metal-coated redox enzymes presented herein can be used in food technology and biotechnology, e.g., for analysis of carbohydrates, organic acids, alcohols, additives, pesticides and fish/meat freshness, in environmental monitoring, e.g., for analysis of pollutants pesticides, and in defense applications, e.g., for detection of chemical warfare agents, toxins, pathogenic bacteria and the likes.

As presented and demonstrated in the Examples section that follows, a metal-coated enzyme was readily absorbed into the screen-printed carbon ink-working electrode. Thus, for glucose-determining electrochemical system, for example, can be based on disposable and multi-arrayed screen-printed electrodes assisted by synthetic mediators such as ferrocene that can react rapidly with the metal-enzyme, and minimize competition with oxygen and other electro-oxidizable species. Screen-printing technology is particularly attractive for the production of disposable sensors, such as for determining glucose levels. The “memory effect” between one sample to another is avoided, and, the phenomenon referred to as “electrode fouling”, which is one of the main drawbacks of the electrochemical sensors, is overcome. Furthermore, these disposable sensors are characterized by high reproducibility and require no calibration.

Screen-printed electrodes are particularly useful in high-throughput screening (HTS) and ultra-high throughput screening (UHTS) technology. Their small size and low cost permit HTS/UHTS of large numbers of electrochemical assays to be conducted simultaneously, at minute volumes of microbiological and/or biochemical samples, using disposable, screen-printed electrochemical microarrays.

Thus, according to preferred embodiments, the electrode used in the glucose biosensor is a screen-printed electrode.

In general, affinity pairs, can be used, for example, for labeling and tagging of bioactive agents, separation techniques such as affinity chromatography, drug delivery and bioactivity screening. In the context of the present invention, metal-coated proteins presented herein can be used as labeling moieties which can be a detectable moiety or a probe when attached to a single or a plurality of various molecules such as bioactive agents, and includes proteins coated with a conductive metal, proteins coated with a radioactive metal, proteins coated with a magnetic metal, as well as any other known detectable metal. Thus, according to embodiments of the present invention, metal-coated proteins presented herein, a detectible metal, can be used for labeling and tagging molecules, cells, tissues, organs and other such bioactive agents directly or indirectly as a part of an affinity-pair system. The indirect labeling is effected via an affinity pair wherein one part of the affinity pair is attached to a detectible metal-coated protein as presented herein, and the second part of the affinity pair is attached to the molecule of interest.

Affinity labeling using the metal-coated proteins can therefore be used for nuclear medicine agents and radiotherapeutics, sensor systems, immunoassays systems, flow cytometry systems, genetic mapping systems, imaging probes, immunohistochemical staining agents, in vivo, in situ and in vitro screening, tracing, localizing and hybridization probes, affinity chromatography agents, magnetic liquids and targeting systems.

The metal-coated proteins of the present invention can be particularly used in imaging techniques which are based on the absorption of energy by heavy metals or the emittance of energy from or radioactive metals.

Hence, according to another aspect of the present invention there is provided an imaging probe which includes the composition-of-matter presented herein, wherein the metal which coats the protein is a detectible metal. Preferably the detectable metal coat includes one or more radioactive isotopes.

In preferred embodiments, the protein is a member of a biologic affinity-pair, as discussed hereinabove, and it's affinity pair counterpart is a part of the tissue and/or the organ to be imaged, therefore the detectible metal can accumulate in these tissues and/or organs specifically and differentially from other tissues and organs which do exhibit the affinity pair counterpart.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

### Materials

Enzyme:

Glucose oxidase from Aspergillus niger (Cat No. G-2133), purchased from Sigma, was selected as an exemplary protein in this study.

Glucose oxidase from Aspergillus niger catalyzes the oxidation of β-D-glucose, producing hydrogen peroxide (H₂O₂) and gluconic acid. Glucose oxidase is a negatively charged dimeric glycoprotein with a molecular weight of 160,000 kD. Inhibitors of glucose oxidase include metal ions, p-chloromercuribenzoate and phenylmercuric acetate [Murachi, T. et al. (1980), Biochimie 62(8-9): 581-5].

The analytical and medicinal importance of this enzyme has been well recognized [see, for example, R. Wilson and A.P.F. Turner, Biosensors & Bioelectronics 1992, 7, pp. 165-185; and N.C. Veitch, Phytochemistry 2004, 65, pp. 249-259]. Glucose oxidase is a glycoprotein having known glycans on its surface, and is characterized by high stability in its isolated and purified form.

Metal:

Palladium (as palladium acetate, Aldrich Cat. No. 20, 586-9 or Pd-chloride, Sigma, Cat. No.: P-0250), purchased from Sigma-Aldrich, was selected as an exemplary metal in view of its abundant and successful use in protein metallization [see, for example, W. Habicht et al. in Surf Interface Anal. 2004, 36, pp. 720-723].

Reducing Agent:

Hypophosphite (HP, Cat. No. 24, 366-3), purchased from Sigma-Aldrich, was selected as an exemplary non-toxic, water soluble and mild reducing agent.

Chelating Agent:

Iminodiacetate (IDA, Cat. No. 23, 487-7), ethylenediamine (EDA, Cat. No. 24, 072-9) and diaminobutane (DAB, Cat. No. 32,790), purchased from Sigma-Aldrich, were selected as exemplary chelating agents.

Reagents:

Glutaraldehyde (GA, Cat. No. 104239) was purchased from Merck.

Polyglutaraldehyde (PGA) was prepared as described by Tor et al. in Enzyme Microb. Technol., 1989, 11, 305-312.

Electrochemical Tests Reagents:

KCl, K₂HPO₄ and KH2PO₄, β-D-glucose were obtained from Merck.

Nafion (5% w/w solution) was purchased from Aldrich.

All solutions were prepared with doubly-distilled water.

High Resolution Transmission Electron Micrographs (HRTEM):

Electron micrographs of the metallic particles on the surface of the metallized enzyme were obtained by a high resolution electron microscope (Philips Tecnai F20) without further staining.

Spectrophotometric Measurements:

The variation in optical density, generated by the formation of solid metallic palladium after reduction of palladium ions, was measured using a spectrophotometer operated at a wavelength of 322 nm.

### Example 1

### Preparation of a Modified Enzyme Having Chelating Moieties Attached to its Surface

Enzyme Modification:

Glucose oxidase (GOX) was modified so as to have free aldehyde groups on its surface, essentially as described by Tor et al. in Enzyme Microb. Technol., 1989, 11, 305-312. The modification is based on reacting polyglutaraldehyde with lysine residues on the enzyme's surface. In brief, GOX enzyme solution (5 ml of a 5 mg/ml stock solution) was incubated at 4° C. overnight in a solution containing polyglutaraldehyde (PGA, 0.076 M) and HEPES buffer (0.05 M, pH=8). Unbound PGA was removed by ultrafiltration, performed by centrifugation using centrifugation tubes (Millipore, Cat. No. UFC805024), to thereby obtain the GOX-PGA modified enzyme.

According to a rough calculation, there are about 30 PGA groups attached to the 30 outwards-pointing lysine residues available for modification in GOX, and each PGA group presents about 10 free aldehyde groups, giving the GOX-PGA modified enzyme about 300 free aldehyde groups.

Enzyme Conjugation with a Diacetate-Chelating Agent:

Iminodiacetate (IDA) is a chelator typically used in immobilized metal affinity chromatography by attaching it to the column resin and utilizing its chelating characteristic to separate metal binging proteins. IDA interacts with divalent metal ions via its acetate groups, so as to form a stable chelating complex at pH range of 5 to 7.

GOX-PGA (4.5 mg/ml) was incubated at 4° C. overnight in a solution (2.5 ml in Hepes buffer 0.05M, pH=8 containing iminodiacetate, 1.4 ml (IDA, 0.25 M) to thereby obtain the GOX-PGA-IDA modified enzyme. Unbound IDA was removed by ultrafiltration, performed by centrifugation using centrifugation tubes, to thereby obtain the GOX-PGA-IDA modified enzyme.

According to a rough calculation, about 300 IDA groups can be attached to a GOX-PGA modified enzyme molecule, which can potentially complex with about 300 divalent metal ions.

Enzyme Conjugation with a Diamine-Chelating Agent

GOX-PGA (4.5 mg/ml) was incubated at 4° C. overnight in a solution (2.5 ml HEPES buffer (0.05 M, pH=8) containing 1.4 ml ethylenediamine (EDA, 0.25 mM) or diaminobutane (DAB, 0.25 Mm) The final concentration of the amine is 0.09 Mm. Unbound EDA or DAB was removed by ultrafiltration, performed by centrifugation using centrifugation tubes, to thereby obtain the GOX-PGA-EDA or GOX-PDA-DAB modified enzyme, respectively.

The general concept for modifying proteins is presented in Scheme 3 below, which depicts a schematic illustration of a protein modified with a schematic polyglutaraldehyde moiety, showing only a part thereof, via an amine group of a lysine residue thereof using the well established Schiff-base (imine) formation reaction. This universal protein modification and conjugation method can be carried out readily under physiological, namely mild conditions. Using similar reaction conditions the conjugation of the chelating moiety, having an amine group, is again effected by forming another imine between the amine and one of the free aldehyde groups present on the PGA, or alternatively via a hydro-amino addition reaction between this amine and the double bond in PGA.

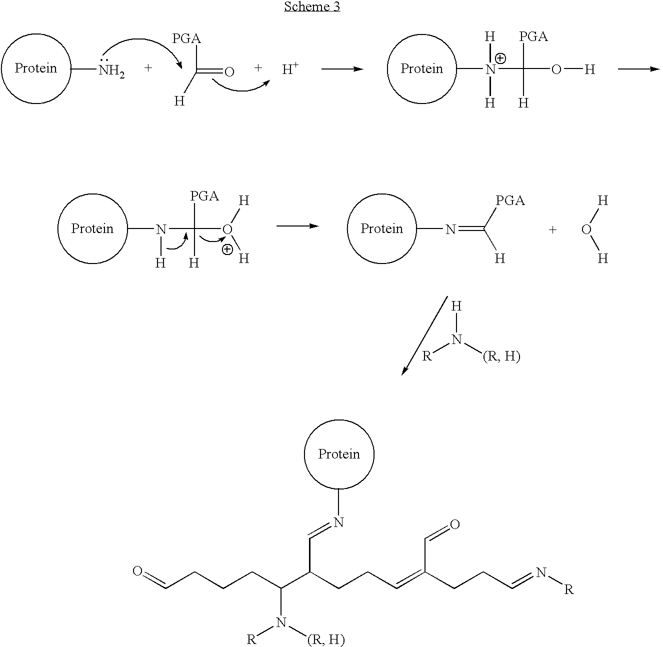

As can be seen in Scheme 3, the PGA moiety introduces a plurality of reactive aldehyde groups to the surface of the protein. Each such aldehyde group can react with, for example, an amine group of a chelating moiety, as depicted in Scheme 3 above, represented by a —N—(R,H)—R group. The R represents the chelating groups (dentates). Hence, a protein modified with PGA and conjugated to IDA, an exemplary bidentate chelating moiety, will have a chemical structure similar to that depicted in Scheme 4 below.

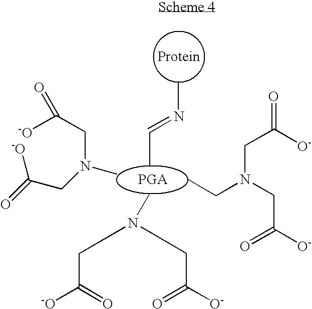

Alternatively, a protein modified with PGA and conjugated to a mixture of EDA and DAB, exemplary bidentate chelating moiety which act as monodentates upon conjugation to the PGA, will have a chemical structure similar to that depicted in Scheme 5 below.

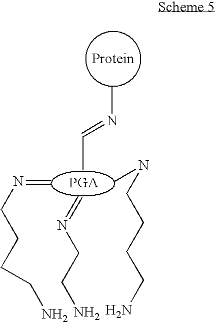

### Example 2

### Preparation of Palladium-Coated Enzyme

Preparation of Glucose Oxidase/Palladium Ion Complex:

Palladium was selected as an exemplary catalytic reduction metal, which would form the oxidative reduction metal coat over the enzyme's surface. The resulting palladium coat can serve as a nucleation site for additional metal atoms upon reacting the Pd-coated enzyme with a solution of other metal ions. Since GOX is a negatively charged protein at neutral pH, positively charged palladium ions could be electrostatically attracted to the enzyme in a neutral solution. The preference of the metal ions to form complexes with the modified enzyme rather than other complexes in solution would depend on the type of metal salt used, the pH of the solution and other components and physical conditions such as temperature and time.

The salt-type dependency was tested by comparing a stable chelator-ion salt such as palladium-ethylenediamine-tetra-acetic acid complex salt (Pd-EDTA) which would allow a controlled release of the palladium ion in solution, and the readily dissociable palladium-acetate salt.

The modified enzyme, GOX-PGA-IDA, GOX-PGA-EDA or GOX-PGA-DAB (4.5 mg/ml), was incubated at room temperature overnight in a solution (0.8 ml) containing 0.8 ml palladium acetate (5 mM) or Pd-chloride (5 mM), and 0.4 ml HEPES buffer (0.05 M, pH=8). Final concentration of the Pd ions is 2 mM. Unbound palladium ions were thereafter removed by ultrafiltration, performed by centrifugation using centrifugation tubes, to thereby obtain a GOX-PGA-IDA-Pd²⁺, a GOX-PGA-EDA-Pd²⁺ or a GOX-PGA-DAB-Pd²⁺ complex, respectively.

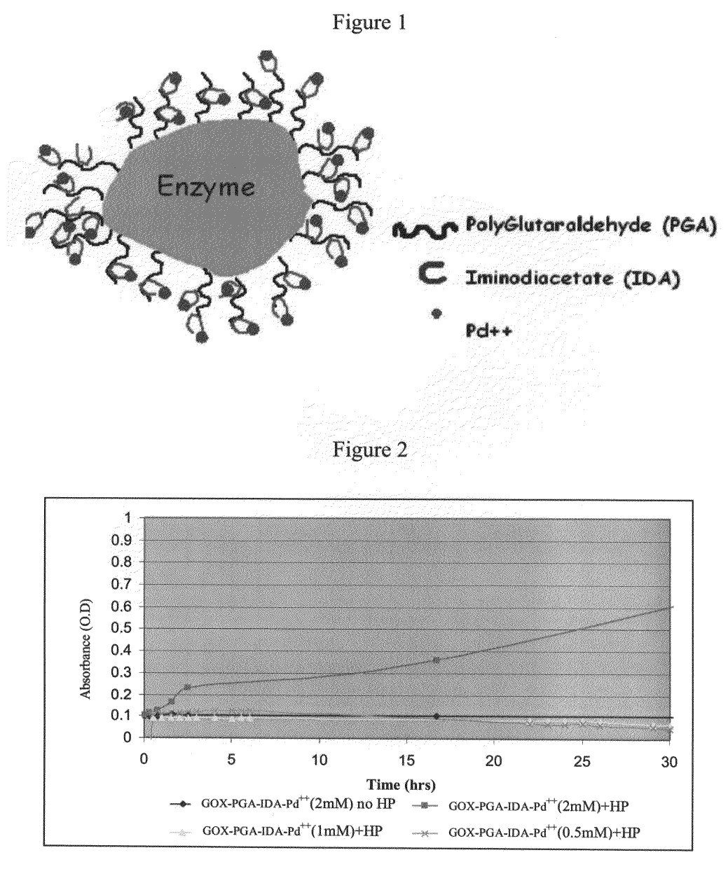

FIG. 1 presents a schematic illustration of the enzyme/metal ion complex obtained using IDA as the chelating moiety (GOX-PGA-IDA-Pd²⁺). As can be seen in FIG. 1, the enzyme (blob-shaped object) is modified by PGA chains (tilde-shaped lines), which are covalently attached to its surface and hence add a plurality of free aldehyde end-groups to the surface of the protein. A plurality of iminodiacetate chelating moieties are attached to the PGA (C-shaped crescents), and form complexation with Pd²⁺ ions (dots).

In-Situ Reduction of Palladium in the Palladium-Glucose Oxidase Complex:

Using the GOX-PGA-IDA-Pd²⁺, a GOX-PGA-EDA-Pd²⁺ or a GOX-PGA-DAB-Pd²⁺ complex, prepared as described above, metallic palladium-coated glucose oxidase was prepared as follows:

The enzyme-bound palladium ions were reduced in-situ by incubating the enzyme-palladium ion complex (4.5 mg/ml)) in a solution (1 ml in HEPES buffer 0.05M ph=8) containing 0.17 ml hypophosphite (0.17 M) and for 10 minutes at room temperature.

The effect of the concentration of palladium ions in the complexing reaction was tested by performing the reaction with three solutions of palladium acetate salt 0.5 mM, 1 mM and 2 mM Pd-acetate concentrations, as described above using an exemplary modified enzyme, namely GOX-PGA-IDA.

These three palladium-glucose oxidase complex samples were reduced with hypophosphite and filtered as described above, and the samples were analyzed spectrophotometrically at a wavelength of 332 nm to quantify the formation of metallic palladium in the samples as a function of time. The results of this study are presented in FIG. 2.

As can be seen in FIG. 2, no change was observed in the tested samples in which low concentrations of palladium ions, namely 0.5 mM and 1 mM Pd-acetate solutions were used. Only the sample in which a high concentration solution of 2 mM Pd-acetate showed a significant and time dependent formation of metallic palladium on the protein's surface.

These results indicate the required palladium ion concentration for forming a stable complex with the modified enzyme. The time dependency coincides with the known autocatalytic reduction process of palladium and/or the migration of palladium atoms and the formation of larger clusters thereof.

Preparation of Palladium-Coated Glucose Oxidase:

After reduction of the palladium ions in the GOX-PGA-IDA-Pd²⁺ complex, additional palladium acetate (0.1 ml, 0.5 mM) was added to the reaction mixture and the metallization (electroless deposition) process was allowed to proceed for 5 hours at room temperature to thereby obtain the palladium-coated GOX. This concentration of 0.5 mM Pd-acetate was selected to demonstrate the feasibility of the process, and was examined for optimization, as described hereinbelow.

The reduction and deposition of the additional palladium atoms onto the GOX-palladium complex was studied as a function of time. The rate of increase of the optical density of the sample due to formation of metallic palladium particles, measured at a wavelength of 332 nm, compared three tested samples, as follows:

Sample 1. GOX-PGA-IDA-Pd²⁺ complex without a reducing agent, denoted “GOX-PGA-IDA-Pd⁺⁺ (No HP)”;

Sample 2. GOX-PGA-IDA-Pd²⁺ complex in the presence of a reducing agent, denoted “GOX-PGA-IDA-Pd⁺⁺+HP”; and

Sample 3. GOX-PGA-IDA-Pd²⁺ complex in the presence of a reducing agent and additional palladium ions (0.5 mM), denoted “GOX-PGA-IDA-Pd⁺⁺+HP+Pd⁺⁺”.

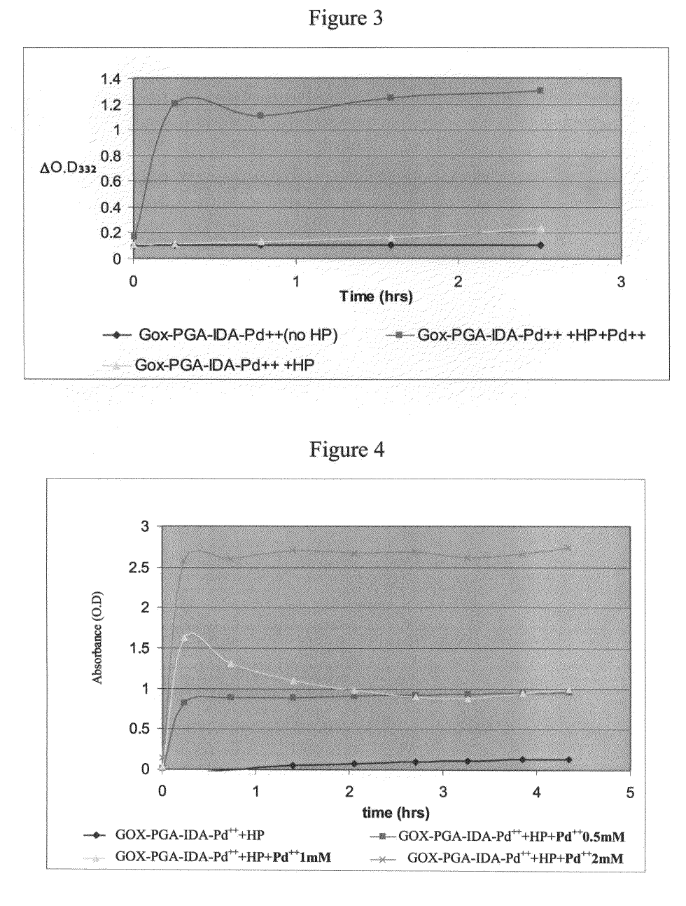

The results of the reduction and deposition of palladium atoms as a function of time are presented in FIG. 3, wherein Sample 1 is marked by blue diamonds, Sample 2 is marked by yellow triangles and Sample 3 is marked by magenta circles.

As can be seen in FIG. 3, Sample 1, containing an enzyme-palladium ions complex in which the palladium ions were not reduced showed no change in the optical density. Sample 2, containing an enzyme-palladium ions complex in which the palladium ions were subjected to reduction in-situ without further treatment with additional palladium ions showed a slight increase in optical density. This slight increase may result from migration of reduced palladium from discrete chelating moieties into fewer clusters of metallic palladium. Sample 3, containing an enzyme-palladium ions complex in which the palladium ions were subjected to reduction in-situ and in which treatment with additional palladium ions was effected showed a sharp increase in optical density in the first quarter of an hour, and an additional slower increase over the next 2.5 hours, clearly indicating that the metallization step of the protein is taking place under the mild conditions and may be completed within about 5 hours.

The effect of the concentration of the added palladium ions on the palladium deposition and coating reaction was also tested by performing the coating reaction using three solutions of palladium acetate at 0.5 mM, 1 mM and 2 mM Pd-acetate concentration, and monitoring change in optical density (ΔOD) spectrophotometrically at a wavelength of 332 nm during the coating process. The results are presented in FIG. 4.

As can be seen in FIG. 4, the concentration of palladium ions used to coat the initial enzyme/palladium complex had an effect on the coating process. Although the fastest onset was observed for the samples coated using the 1 mM Pd-acetate solution, it seems that in the course of time the ΔOD of the samples coated using the 0.5 mM and the 1 mM Pd-acetate solutions leveled while the ΔOD of the sample coated using the 2 mM Pd-acetate solution steadily increased and reached higher levels.

FIG. 5 presents a metallic palladium patch which was deposited on the surface of a glucose oxidase molecule, upon treating an enzyme-palladium ions complex with a reducing agent and additional palladium ions, similar to Sample 3 above using a 0.5 mM Pd-acetate solution for the coating process, as seen in a high resolution electron micrograph microscope obtained without staining.

As can be seen in FIG. 5, a patch of deposited palladium of about 10 nm in diameter is clearly visible on the surface of the glucose oxidase, having a disordered or partially crystalline morphology.

In order to verify that the palladium patches such as the one observed and presented in FIG. 5, are deposited on the surface of the enzyme, thus forming an enzyme/palladium hybrid, the patches were chemically analyzed using electron dispersion spectroscopy (EDS), as presented in FIG. 6.

As can be seen in FIG. 6, the chemical analysis corroborates that the observed patched are indeed of palladium. The spectrograph also shows peaks of carbon and oxygen stemming from the protein, and peaks of phosphorous stemming from the reducing agent. The copper peak stems from the sample microgrid.

### Example 3

### Preparation of Nickel-, Cobalt- and Copper-Coated Glucose Oxidase

The possibility to coat GOX with other metals was examined for cobalt, nickel and copper. These metals have various physical and chemical properties which can open new and varied avenues of applications, such as increased electrical and heat conductivity, acquired magnetism for localization and targeting, biocidal activity and potential biochemical targeting and imaging thereof. These metals were selected also to demonstrate the possibility of coating the enzyme with metals having different standard electrode potentials by electroless deposition.

The standard electrode potentials for the metals used in this example are listed below:

Pd2⁺+2e⁻→Pd⁰ +0.915 E°/V;

Cu²⁺+2e^{-→}Cu⁰ +0.340 E°/V;

2H⁺+2e^{-→}H₂ 0 E°/V reference;

Co²⁺+2e⁻→Co⁰ −0.277 E°/V; and

Ni²⁺+2e⁻→Ni⁰ −0.257 E°/V.

Using the GOX-PGA-IDA-Pd²⁺, GOX-PGA-EDA-Pd²⁺ or GOX-PGA-DAB-Pd²⁺ complexes, prepared as described above in Example 2, metallic nickel-, cobalt- or copper-coated glucose oxidase was prepared by first preparing a series of electroless-deposition (ELD) solutions containing glycine (0.49 M), H₃BO₃ (0.5 M), and either nickel, cobalt or copper chloride (18 mM, 2 mM or 0.5 mM), and adjusting the solution to pH=7. Thereafter the enzyme-bound palladium ions were reduced in-situ by incubating the enzyme-palladium ion complex (4.5 mg/ml) in a solution (1 ml in HEPES buffer (0.05 M, pH=8) containing 0.17 ml hypophosphite (0.17 M) for 10 minutes at room temperature. Once the palladium was reduced, an ELD solution (1 ml, 10 mM) containing nickel, cobalt or copper was added and the reaction was allowed to proceed for 5 hours at room temperature, to thereby obtain the nickel-, cobalt- or copper-coated GOX. The final concentration of the metals was 5 mM.

As with the palladium-coated enzyme, the copper, cobalt and nickel-coated enzyme samples were analyzed by HRTEM, and the obtained micrographs are presented in FIGS. 7A-F.

As can be seen in FIG. 7, the copper-coated enzyme samples (FIGS. 7A and 7B) exhibited round metal patches in the range of 10 nm to 20 nm in diameter, having an amorphous morphology. The cobalt-coated enzyme samples (FIGS. 7C and 7D) exhibited round metal patches in the range of 5 nm to 20 nm in diameter, having a crystalline morphology. The nickel-coated enzyme samples (FIGS. 7E and 7F) also exhibited round metal patches but their diameter and morphology were undefined.

### Example 4

### Enzymatic Activity and Dissolvability of Metal-Coated Enzymes

Enzymatic Activity and Dissolvability of Palladium-Coated Glucose Oxidase:

The effect of palladium deposition on the enzymatic activity of the palladium-coated GOX enzyme obtained by the process presented hereinabove (see, Example 2) was studied by measuring the specific activity of native (untreated) glucose oxidase, and comparing it to the residual specific activity of the enzyme after each step of the process for obtaining the palladium-coated enzyme.

The activity assays were performed as previously described by Nakai et al. [J. Phys. Chem. B 2001, 105, 1701-1704].

The effect of palladium deposition on the dissolvability of the untreated and palladium-coated enzymes was evaluated visually.

The following samples were used in these activity and dissolvability assays:

1. Untreated glucose oxidase, denoted “GOX— untreated”;

2. Enzyme modified with polyglutaraldehyde, denoted “GOX-PGA”;

3. Enzyme modified with polyglutaraldehyde and conjugated to iminodiacetate, denoted “GOX-PGA-IDA”;

4. Enzyme-palladium ion complex, denoted “GOX-PGA-IDA-Pd⁺⁺ No HP”;

5. Palladium ions and hypophosphite, denoted “Pd⁺⁺+HP (no GOX)”;

6. Enzyme-metallic palladium complex, denoted “GOX-PGA-IDA-Pd⁺⁺+HP”; and

7. Palladium-coated glucose oxidase, denoted “GOX-PGA-IDA-Pd⁺⁺+HP+Pd⁺⁺”.

The obtained results are presented in Table 3 below.

TABLE 3

| | | | % of Residual | |
|---|---|---|---|---|
| Entry | Assayed Sample | U/mg | Specific activity | Dissolvability |
| 1 | GOX - untreated | 100.75 | 100 | CLEAR |
| 2 | GOX-PGA | 71.10 | 71 | CLEAR |
| 3 | GOX-PGA-IDA | 66.72 | 66 | CLEAR |
| 4 | GOX-PGA-IDA-Pd⁺⁺ No HP | 23.78 | 24 | CLEAR |
| 5 | Pd⁺⁺ + HP (no GOX) | N/A | N/A | PRECIPITATE |
| 6 | GOXGOX-PGA-IDA-Pd⁺⁺ + HP | 43.80 | 46 | CLEAR |
| 7 | GOX-PGA-IDA-Pd⁺⁺ + HP + Pd⁺⁺ | 41.75 | 46 | CLEAR |

The activity and dissolvability of the native (untreated) enzyme are presented in entry 1 of Table 3, and serve as a control standard for enzymatic activity and dissolvability to which the results obtained for the treated enzyme sample are compared. A sample containing palladium ions and the reducing agent hypophosphite (actually containing reduced, metallic, palladium), presented in entry 5 of Table 3, resulting in metallic palladium, served as a qualitative control sample for the dissolvability assay.

As can be seen in Table 3, the assay conducted for the PGA-modified and IDA-conjugated enzyme, presented in entries 2 and 3 of Table 3 respectively, showed a moderate decrease in specific activity, as expected from a chemically modified protein. On the other hand, the enzyme/palladium ion complex (unreduced palladium), presented in entry 4 of Table 3, showed a decrease of 76% of the specific activity of the enzyme. The inhibition of enzymatic activity can be attributed to the presence of metal ions, which are known as effective inhibitors of GOX.

As can further be seen in Table 3, the assay conducted for the enzyme/metallic palladium complex, presented in entry 6 of Table 3, and the assay conducted for the palladium-coated enzyme, presented in entry 7 of Table 3, showed a considerable retention of 46% of the specific activity of the enzyme, indicating that once the metal ions are reduced to elemental metal atoms, possibly because they no longer inhibit the enzyme to the extent seen in entry 6, and that the deposition of additional metallic coat on the surface of the enzyme of entry 7 does not diminish the enzyme's activity, below the activity of the enzyme presented in entry 6.

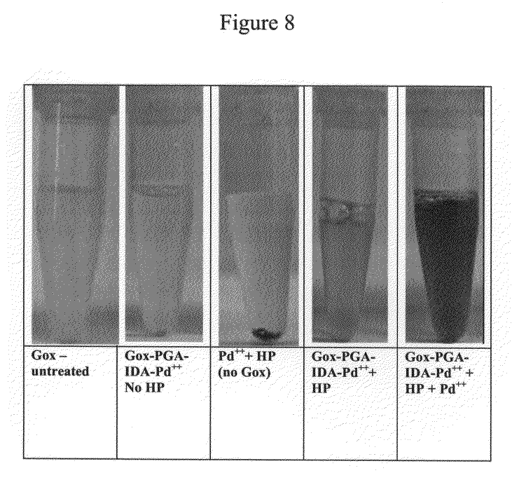

The results of the visual dissolvability assay of the above samples are presented in FIG. 8.

As can be seen in FIG. 8, the samples wherein the palladium ions are not present, as in the samples denoted “GOX—untreated”, or wherein the palladium ions are not reduced, as in the samples denoted “GOX-PGA-IDA-Pd⁺⁺No HP”, remained clear and substantially untinted. The qualitative control sample denoted “Pd⁺⁺+HP (no GOX)” showed the expected result of reducing palladium ions into metallic palladium, namely the formation of insoluble metallic particles and precipitation thereof at the bottom of the test-tube.

As can further be seen in FIG. 8, both the sample wherein the palladium ions are reduced in-situ on the protein, as in the samples denoted “GOX-PGA-IDA-Pd⁺⁺+HP”, and the sample wherein additional palladium ions are reduced and deposited on the surface of the enzyme, as in the samples denoted “GOX-PGA-IDA-Pd⁺⁺+HP+Pd⁺⁺”, exhibited the expected formation of a dark tint attributed to the metallic palladium atoms or atom clusters on the enzyme surface. Yet, the lack of precipitation indicated that the palladium atoms form a part of a soluble protein/metal complex, and further showed that even the metal-coated enzyme sample, having a thickened layer of metallic palladium deposited on the surface of the protein, remained soluble.

These results clearly indicate that using the methodologies for depositing palladium on the surface of enzymes, described hereinabove, palladium-coated glucose oxidase, which retains almost 46% of its native activity, and substantially maintains its dissolvability, can be achieved.

Enzymatic Activity and Dissolvability of Copper-, Cobalt- or Nickel-Coated Glucose Oxidase

The effect of copper, nickel and cobalt deposition at various concentrations on the enzymatic activity of the metal-coated GOX enzyme obtained by the process presented hereinabove (see, Example 3) was studied by measuring the specific activity of native (untreated) glucose oxidase, and comparing it to the residual specific activity of the enzyme after each step of the process for obtaining the metal-coated enzyme, and examining the effect of the concentration of the electroless deposition metal ion solution (ELD).

The following samples were used in these activity assays:

1. Untreated glucose oxidase, denoted “GOX—untreated”;

2. Enzyme modified with polyglutaraldehyde, denoted “GOX-PGA”;

3. Enzyme modified with polyglutaraldehyde and conjugated to iminodiacetate, denoted “GOX-PGA-IDA”;

4. Enzyme-metallic palladium complex, denoted “GOX-PGA-IDA-Pd⁺⁺+HP”; and

5. Copper-coated glucose oxidase, prepared using a 0.5 mM copper salt ELD solution, denoted “GOX-PGA-IDA-Pd⁺⁺+HP+Cu⁺⁺”.

6. Copper-coated glucose oxidase, prepared using a 2 mM copper salt ELD solution, denoted “GOX-PGA-IDA-Pd⁺⁺+HP+Cu⁺⁺”.

7. Cobalt-coated glucose oxidase, prepared using a 0.5 mM cobalt salt ELD solution, denoted “GOX-PGA-IDA-Pd⁺⁺+HP+Co⁺⁺”.

8. Cobalt-coated glucose oxidase, prepared using a 2 mM cobalt salt ELD solution, denoted “GOX-PGA-IDA-Pd⁺⁺+HP+Co⁺⁺”.

9. Nickel-coated glucose oxidase, prepared using a 0.5 mM nickel salt ELD solution, denoted “GOX-PGA-IDA-Pd⁺⁺+HP+Ni⁺⁺”.

10. Nickel-coated glucose oxidase, prepared using a 2 mM nickel salt ELD solution, denoted “GOX-PGA-IDA-Pd⁺⁺+HP+Ni⁺⁺”.

The obtained results are presented in Table 4 below.

TABLE 4

| | | % of Residual |
|---|---|---|
| No. | Hybrid type | Specific activity |
| 1 | GOX - untreated | 100 |
| 2 | GOX-PGA | 71 |
| 3 | GOX-PGA-IDA | 66 |
| 4 | GOX-PGA-IDA-Pd⁺⁺ + HP | 46 |
| 5 | GOX-PGA-IDA-Pd⁺⁺ + HP + Cu⁺⁺(0.5 mM) | 28 |
| 6 | GOX-PGA-IDA-Pd⁺⁺ + HP + Cu⁺⁺(2 mM) | 4 |
| 7 | GOX-PGA-IDA-Pd⁺⁺ + HP + Co⁺⁺(0.5 mM) | 32 |
| 8 | GOX-PGA-IDA-Pd⁺⁺ + HP + Co⁺⁺(2 mM) | 38 |
| 9 | GOX-PGA-IDA-Pd⁺⁺ + HP + Ni⁺⁺(0.5 mM) | 40 |
| 10 | GOX-PGA-IDA-Pd⁺⁺ + HP + Ni⁺⁺(2 mM) | 39 |

As can be seen in Table 4, the results of the activity assay show similar residual activity as measured for the palladium-coated GOX, presented hereinabove, namely a residual activity which ranges between about 30 to about 40%. It is also seems that the inactivation or inhibition of GOX does not depend on the type of metal and the concentration of its salt, as similar residual activities were measured for cobalt and nickel, at both ELD solution salt concentrations, namely 0.5 mM and 2 mM. Outstanding was the copper-coated enzyme which seems to lose most of its activity at an ELS solution concentration of 2 mM. This result coincides with the fact that Cu++ ions are known to inhibit GOX, but the fact that all the metal-coated enzyme samples were thoroughly washed and filtered off of metal ions, the assumption is that the difference in the activity noted for copper stems from differences in the way the enzyme was coated, namely the thickness of the coat and the coverage of the surface of the enzyme.

The results presented in Table 4 demonstrate again the feasibility and flexibility of the concept presented herein, of metal-coating an enzyme while retaining a significant percentage of its original activity.

### Example 5

### Electrochemical Activity of Metal-Coated Enzymes

The electrochemical activity of electrode-bound GOX is an alternative procedure to compare the metal-coated enzyme to the native enzyme, and thus evaluate the effect of the conductive coating on the enzyme.

The experiment is effected by measuring the current of an electrochemical cell having GOX immobilized onto a working electrode while applying a linearly alternating positive to negative potential, reintroducing the substrate, glucose, into the reaction cell at each reiteration, and using ferrocene (Fc) as an electron transfer mediator.

Preparation of Enzyme Electrode:

A platinum disk-shaped electrode (2 mm in diameter) embedded in Teflon was polished with 0.3 μm alumina, washed with doubly-distilled water, and thereafter immersed for 10 minutes in a sonicator bath, followed by washing in doubly-distilled water. Native GOX, palladium-coated GOX or cobalt-coated GOX enzyme solutions (2 μl, 3 mg/ml) in HEPES buffer (0.05 M, pH=8) were deposited onto the platinum electrode and allowed to dry at room temperature. Thereafter, the enzyme electrodes were covered with nafion (2 μl, diluted to 0.05% with doubly-distilled water) and allowed to dry at room temperature.

Electrochemical Measurements:

All measurements were performed using a BAS potentiostat (Bio-Analytical Systems, US). The electrochemical cell contained three electrodes: a Pt-modified working electrode having the enzyme applied thereon, a platinum wire counter electrode and an Ag/AgCl reference electrode. The voltammogram measurements were recorded while stirring at a constant speed of 100 rpm using a magnetic stirrer. All experiments were carried out at room temperature.

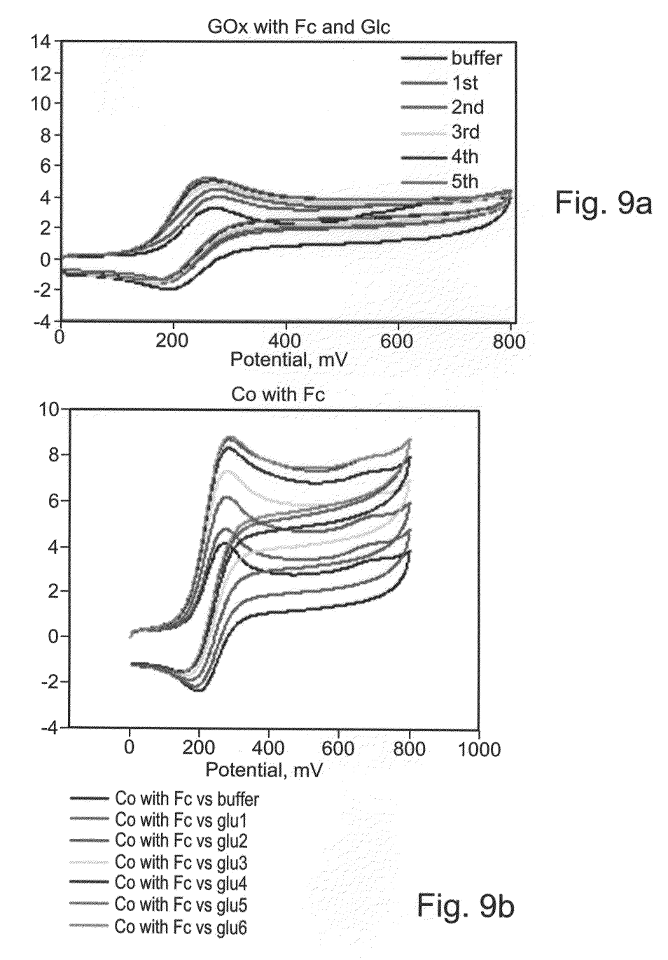

FIG. 9 presents comparative plots of cyclic voltammograms of electro-catalytic currents (in microamperes) plotted versus electric potential (in millivolts) as recorded in five reiterations for a sample of native glucose-oxidase (FIG. 9A), and a similar plot as recorded in six reiterations for a sample of cobalt-coated glucose-oxidase (FIG. 9B).

As can be seen in FIG. 9, the current peaks recorded for the cobalt-coated GOX are significantly higher than the current peaks recorded for the control native enzyme, thus indicating an improved electron transfer in the system, probably due to the conductive coat over the enzyme.

Chronoamperometric experiments with glucose were conducted at constant applied potential of +600 mV in phosphate buffer (0.1 M, pH=5.8) with KCl (0.1 M), that was stirred during measurements at a constant speed of 100 rpm using a magnetic stirrer. All experiments were carried out at room temperature.

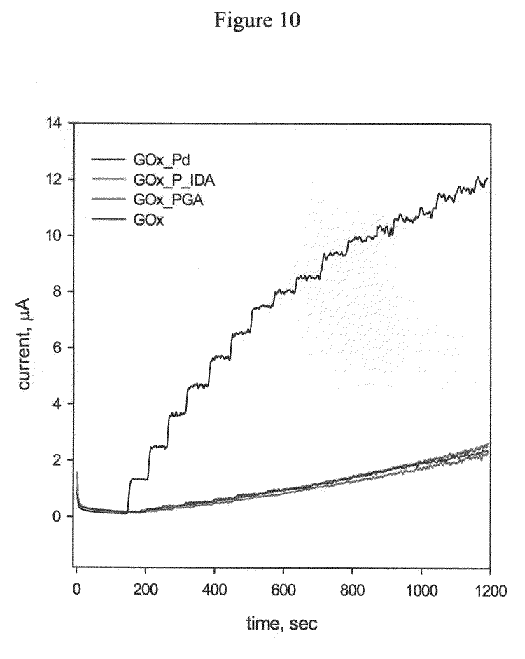

FIG. 10 presents comparative chronoamperometric plots recorded for a modified working electrode having deposited thereon untreated glucose oxidase (blue line), polyglutaraldehyde-treated glucose oxidase (green line), PGA and IDA-treated glucose oxidase (red line), and PGA and IDA-treated glucose oxidase coated with palladium (black line).

As can be seen in FIG. 10, the electrode having deposited thereon a metal-coated enzyme exhibited enhanced electrochemical activity as compared to the almost electrochemically inactive samples of the uncoated enzyme samples.

### Example 6

### Metal-Coated Bacterial Cells

E. coli/Palladium Hybrids:

Washed cells (E. coli strain MG1655) suspended in 1 ml ice cold phosphate buffer (PBS) were added to a polyglutaraldehyde solution (5 ml, 0.5% PGA) in PBS at 4° C. and allowed to incubate therein overnight. Thereafter the PGA-treated cells were harvested by centrifugation (5000 rpm, 10 minutes), washed, resuspended in 1 ml PBS solution and added into a solution of EDA or DAB (5 ml, 0.09 mM) in PBS at 4° C. and the mixture was incubated overnight. The PGA-EDA/DAB-treated cells were harvested by centrifugation (5000 rpm, 10 minutes), washed and resuspended in saline (1 ml of 0.9% NaCl).

Activated cells in 1 ml saline, displaying EDA or DAB chelating moieties, were incubated with palladium acetate solution (5 ml, 2 mM in 0.9% NaCl), at room temperature, overnight. Unbound palladium ions were removed by centrifugation filtration.

The palladium-coated cells (E. coli-PGA-EDA/DAB-Pd⁺⁺) were thereafter incubated in 5 ml of a solution of 0.17 M hypophosphite solution in NaCl 0.9% for 3 hours at room temperature. The cells were harvested by centrifugation (5000 rpm, 10 minute), washed and resuspended in 1 ml NaCl 0.9% solution. Palladium acetate solution (0.005 mM) was thereafter added and the reaction was allowed to proceed for 2 hours at room temperature. Unreduced palladium ions were removed by centrifugation filtration.

The thus prepared coated cells were analyzed by HRTEM. The obtained images demonstrated the presence of round palladium patches on the cells surface (data not shown).

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1-50. (canceled)

51. A composition-of-matter comprising a protein having a surface and a metal coating deposited over at least a portion of said surface and forming a metal-coated protein being dissolvable or suspendable in an aqueous medium, said metal being selected from the group consisting of a single metal and a combination of at least two metals, said single metal being devoid of silver, said metal coating consisting of elemental metal atoms.

52. The composition-of-matter of claim 51, wherein said protein has a biological activity and said metal-coated protein retains said biological activity.

53. The composition-of-matter of claim 51, wherein said metal-coated protein is prepared by contacting a modified protein having at least one chelating moiety attached to said surface with a reducing agent, said chelating moiety being for forming a complex with ions of said metal.

54. The composition-of-matter of claim 51, wherein said metal coating comprises at least one continuous metal particle having a size that ranges from about 5 nm in diameter to about 50 nm in diameter.

55. The composition-of-matter of claim 51, wherein a molar ratio between the protein and the metal ranges from about 1:10 to about 1:10000.

56. A composition-of-matter comprising a protein having a surface and further having a biological activity and a metal coating deposited over at least a portion of said surface and forming a metal-coated protein retaining said biological activity, said metal being selected from the group consisting of a single metal and a combination of at least two metals, said single metal being devoid of silver, said metal coating consisting of elemental metal atoms.

57. The composition-of-matter of claim 56, wherein said metal-coated protein is dissolvable or suspendable in an aqueous medium.

58. The composition-of-matter of claim 56, wherein said metal-coated protein is prepared by contacting a modified protein having at least one chelating moiety attached to said surface with a reducing agent, said chelating moiety being for forming a complex with ions of said metal.

59. The composition-of-matter of claim 56, wherein said metal coating comprises at least one continuous metal particle having a size that ranges from about 5 nm in diameter to about 50 nm in diameter.

60. The composition-of-matter of claim 56, wherein a molar ratio between the protein and the metal ranges from about 1:10 to about 1:10000.

61. A composition-of-matter comprising a protein having a modified surface and a metal coating deposited over at least a portion of said surface and forming a metal-coated protein, said modified surface having at least one chelating moiety attached thereto, said chelating moiety being for forming a complex with ions of said metal, said metal coating consisting of elemental metal atoms.

62. The composition-of-matter of claim 61, wherein said metal coating comprises at least one continuous metal particle having a size that ranges from about 5 nm in diameter to about 50 nm in diameter.

63. The composition-of-matter of claim 61, wherein a molar ratio between the protein and the metal ranges from about 1:10 to about 1:10000.

64. The composition-of-matter of claim 61, wherein said protein has a biological activity and said metal-coated protein retains said biological activity.

65. The composition-of-matter of claim 61, wherein said metal-coated protein is dissolvable or suspendable in an aqueous medium.

66. A process of preparing a metal-coated protein, the process comprising:
reacting the protein with at least one chelating moiety, to thereby obtain a modified protein having said chelating moiety attached to at least a portion of a surface thereof, said chelating moiety being for forming a complex with ions of the metal,
contacting said modified protein with a first aqueous solution containing ions of said metal to thereby obtain a solution containing a complex of said modified protein and said metal ions; and
contacting said solution containing said complex of said modified protein and said metal ions with a first reducing agent, said first reducing agent being for reducing said ions of said metal, thereby obtaining the metal-coated protein.

67. The process of claim 66, further comprising, subsequent to or concomitant with said contacting with said first reducing agent:
contacting the metal-coated protein or said solution containing said complex, with a second aqueous solution containing a plurality of ions of a second metal, in the presence of a second reducing agent, said second reducing agent being for reducing said ions of said second metal, to thereby obtain the metal-coated protein having an additional coating of said second metal on said surface.

68. The process of claim 66, wherein reacting said protein with said at least one chelating moiety comprises:
modifying at least a portion of a surface of the protein, to thereby obtain a modified protein having a plurality of reactive groups on said surface; and
conjugating to at least a portion of said reactive groups said chelating moiety.

69. A pharmaceutical composition comprising, as an active ingredient, the composition-of-matter of claim 51 and a pharmaceutically acceptable carrier.

70. The pharmaceutical composition of claim 69, being packaged in a packaging material and identified in print, in or on said packaging material, for use in the treatment of a bacterial and/or fungal infection.

71. A pharmaceutical composition comprising, as an active ingredient, the composition-of-matter of claim 56 and a pharmaceutically acceptable carrier.

72. The pharmaceutical composition of claim 71, being packaged in a packaging material and identified in print, in or on said packaging material, for use in the treatment of a bacterial and/or fungal infection.

73. A pharmaceutical composition comprising, as an active ingredient, the composition-of-matter of claim 61 and a pharmaceutically acceptable carrier.

74. The pharmaceutical composition of claim 73, being packaged in a packaging material and identified in print, in or on said packaging material, for use in the treatment of a bacterial and/or fungal infection.

75. A method of treating a bacterial and/or fungal infection, the method comprising administering to a subject in need thereof a therapeutically effective amount of the composition-of-matter of claim 51.

76. A method of treating a bacterial and/or fungal infection, the method comprising administering to a subject in need thereof a therapeutically effective amount of the composition-of-matter of claim 56.

77. A method of treating a bacterial and/or fungal infection, the method comprising administering to a subject in need thereof a therapeutically effective amount of the composition-of-matter of claim 61.

78. A metallic element comprising a composition-of-matter which comprises a protein having a surface and a metal coating deposited over at least a portion of said surface and forming a metal-coated protein, said metal being selected from the group consisting of a single metal and a combination of at least two metals, said single metal being devoid of silver, said metal coating consisting of elemental metal atoms.

79. The metallic element of claims 78, wherein said metal in said metal-coated protein is a conductive metal or a semi-conductive metal.

80. An electronic circuit assembly comprising an arrangement of conductive elements interconnecting a plurality of electronic elements wherein at least a portion of said conductive elements comprises the metallic element of claim 79.

81. A device comprising a plurality of the metallic elements of claim 78.

82. An electrode comprising a composition-of-matter deposited thereon, said composition-of-matter comprises a protein having a surface and a metal coating deposited over at least a portion of said surface and forming a metal-coated protein, said metal being selected from the group consisting of a single metal and a combination of at least two metals, said single metal being devoid of silver, said metal coating consisting of elemental metal atoms.

83. A biosensor system for electrochemically determining a level of an analyte in a liquid sample, the system comprising:
an insulating base; and
an electrode system which comprises the electrode of claim 82, wherein said protein is selected capable of chemically reacting with the analyte while producing a transfer of electrons.

84. A method of electrochemically determining a level of an analyte in a liquid sample, the method comprising:
contacting the biosensor system of claim 83 with the liquid sample; and
measuring said transfer of electrons, thereby determining the level of the analyte in the sample.

85. An imaging probe comprising a composition-of-matter which comprises a protein having a surface and a metal coating deposited over at least a portion of said surface and forming a metal-coated protein, said metal being selected from the group consisting of a single metal and a combination of at least two metals, said single metal being devoid of silver, at least one of said metals being a detectable metal, said metal coating consisting of elemental metal atoms.
